(19) United States
(12) Patent Application Publication
(10) Pub. No.: US 2026/0158087 A1
(43) Pub. Date: Jun. 11, 2026

(54) HERPESVIRAL COMBINATION THERAPY FOR TARGETING CANCER CELLS AND CANCER ASSOCIATED STROMAL CELLS

(71) Applicant: Mayo Foundation for Medical Education and Research, Rochester, MN (US)

(72) Inventors: Haifei Jiang, Rochester, MN (US); Stephen James Russell, Rochester, MN (US)

(21) Appl. No.: 18/707,349

(22) PCT Filed: Nov. 4, 2022
(86) PCT No.: PCT/US2022/048950

§ 371 Date: May 3, 2024

Related U.S. Application Data
(60) Provisional application No. 63/275,727, filed on Nov. 4, 2021.

(51) Int. Cl. A61K 35/763; A61P 35/00

(52) U.S. Cl. CPC A61K 35/763; A61P 35/00

(57) ABSTRACT

Methods and materials for targeting cancer cells and cancer-associated stromal cells within a tumor are provided herein. For example, this document provides compositions that contain recombinant herpesviruses from two or more different sub-families, and also provides methods for using such compositions to treat mammals having cancer.

### CROSS-REFERENCE TO RELATED APPLICATIONS

This application claims the benefit of U.S. Provisional Application Ser. No. 63/275,727, filed Nov. 4, 2021. The disclosure of the prior application is considered part of (and is incorporated by reference in) the disclosure of this application.

### TECHNICAL FIELD

This document relates to methods and materials for targeting cancer cells and cancer-associated stromal cells within a tumor. For example, this document relates to compositions that contain recombinant herpesviruses from two or more different sub-families, and to methods for using such compositions to treat mammals having cancer.

### BACKGROUND

The human herpesvirus family has nine members that belong to the α, β, and γ sub-families. Each herpesvirus member presents distinct cell tropism in infected tissues due to cellular receptor selection and differential intracellular replication kinetics. For example, the alpha herpesviral herpes simplex virus (HSV-1) and varicella-zoster virus (VZV) are epithelial and neuronal tropic, while the cell tropism of beta herpesviral cytomegalovirus (CMV) (myeloid, fibroblast and endothelial tropic) is completely different from that of the alpha herpesviruses.

### SUMMARY

This document is based, at least in part, on the development of human herpesviral combination therapies for treating tumors. The natural tropisms and replication characteristics of the three human herpesviruses, HSV-1, CMV, and VZV, were used, and virus templets were designed, by adding reporter genes, mutating viral genes, enhancing virus spread and immunogenic cell death, and inserting boosting transgenes. The complementary viral tropisms and characteristics between alpha (e.g., HSV and VZV) and beta (e.g., CMV) herpesviruses were combined to increase oncolytic effects. The viruses were tested as monotherapies or combination therapies in in vivo tumor models to evaluate oncolytic properties and mechanisms.

As described herein, combining herpesviruses from different sub-families can be useful to target both cancer cells and cancer-associated stromal cells within a tumor, inducing synergistically enhanced anti-tumor responses. This document provides, for example, materials and methods for using herpesviruses from different sub-families (e.g., alpha herpesviruses such as HSV-1 or VZV, and beta herpesviruses such as CMV) to target both tumor cells and associated stromal cells. As demonstrated herein, recombinant HSV-1, VZV, and CMV constructs were generated based on a bacterial artificial chromosome (BAC)-lambda-red recombineering system, with human sodium iodide symporter (hNIS) and human chorionic gonadotropin beta subunit (β-hCG) sequences inserted into the backbone of an HSV-1 KOS strain as reporter genes to track the level and the location of virus replication in vivo. In two subcutaneous syngeneic models (A20 and EMT6), a significant improvement in anti-tumor efficacy was observed with the combination of a HSV-1 strain (e.g., KOS-BAC NIS-hCG ΔICP47) and a murine CMV (mCMV) Smith strain. The HSV-1 and mCMV constructs targeted different cell types within the injected tumors, and the myeloid tropism of CMV was confirmed. For VZV vectors, the β-hCG gene was inserted into the genome of the live-attenuated vaccine Oka strain as a reporter gene, and a hyperfusogenic gBY881F mutation was made to enhance virus-induced intracellular cell fusion. Testing in the human MeWo melanoma xenograft model confirmed a potent anti-tumor efficacy for the vOka-BAC virus.

The herpesviral combination therapy platform described herein can be used to target both cancer and stromal cells, and to concurrently express multiple tumor microenvironment modulating factors for enhancing virus induced anti-tumor immune responses. The methods and materials provided herein provide advantages over existing technologies that utilize viruses to treat cancer. For example, most existing technologies have been designed to specifically infect cancer cells, while leaving the uninfected tumor stromal cells to be actively involved in removing virus infection and secreting pro-survival signals. In contrast, using the method and materials provided herein, both cancer cells and associated stromal cells can be infected, and their phenotype can be shifted from pro-tumor to anti-tumor. In addition, the herpesviral combination therapy platform described herein can be used to induce synergistic anti-tumor effects in the tumor microenvironment, and more therapeutic transgenes can be expressed.

In a first aspect, this document features a composition containing, consisting essentially of, or consisting of a first recombinant human herpesvirus and a second recombinant human herpesvirus, where the first and second recombinant human herpesviruses are from two different herpesvirus sub-families, and where, when the composition is administered to a mammal having cancer, the first recombinant human herpesvirus infects a cancer cell and the second recombinant human herpesvirus infects a stromal cell associated with the cancer, thereby reducing the number of cancer cells within the mammal.

The first recombinant human herpesvirus can be a human alpha herpesvirus. The human alpha herpesvirus can be a recombinant human HSV or a recombinant human VZV. For example, the human alpha herpesvirus can be a recombinant human HSV. The recombinant human HSV can (a) lack both copies of ICP34.5, (b) lack functional ICP47, (c) contain nucleic acid encoding a mutant gB polypeptide having hyperfusogenic activity, or (d) contain a heterologous nucleic acid sequence encoding a therapeutic polypeptide. In some cases, the recombinant human HSV (a) lacks both copies of ICP34.5, or (b) lacks functional ICP47. In some cases, the recombinant human HSV lacks both copies of ICP34.5. In some cases, the recombinant human HSV lacks functional ICP47. The recombinant human HSV can contain nucleic acid encoding the mutant gB polypeptide. The recombinant human HSV can contain the heterologous nucleic acid sequence encoding a therapeutic polypeptide. The therapeutic polypeptide can be selected from the group consisting of pigment epithelium-derived factor (PEDF), CCL4, CXCL13, IL-12, IL-15, and brain-derived neurotropic factor (BDNF). The recombinant human HSV can (a) lack both copies of ICP34.5 or lacks functional ICP47, (b) contain nucleic acid encoding a mutant gB polypeptide having hyperfusogenic activity, and (c) contain a heterologous nucleic acid sequence encoding a therapeutic polypeptide (e.g., a therapeutic polypeptide is selected from the group consisting of PEDF, CCL4, CXCL13, IL-12, IL-15, and BDNF). The recombinant human HSV can contain a cell type specific promoter sequence that drives expression of an essential viral gene of the recombinant human HSV within the cancer cell. The recombinant human HSV can contain nucleic acid encoding a single-chain variable fragment (scFv) fused to an HSV glycoprotein. The scFv can be capable of binding to a polypeptide expressed by the cancer cell. The recombinant human HSV can contain nucleic acid encoding a reporter polypeptide. The reporter polypeptide can be selected from the group consisting of human sodium/iodide symporter (hNIS) and human chorionic gonadotropin beta subunit (beta-hCG).

The human alpha herpesvirus can be a recombinant human VZV. The recombinant human VZV can (a) be attenuated via natural genetic variability, an induced mutation, or reverse genetics or (b) contain a heterologous nucleic acid sequence encoding a therapeutic polypeptide. For example, in some cases, the recombinant human VZV is attenuated via natural genetic variability, an induced mutation, or reverse genetics. In some cases, the recombinant human VZV is attenuated via an induced mutation. In some cases, recombinant human VZV contains the heterologous nucleic acid sequence encoding a therapeutic polypeptide. The therapeutic polypeptide can be selected from the group consisting of PEDF, CCL4, CXCL13, IL-12, IL-15, and BDNF. The recombinant human VZV can contain nucleic acid encoding a mutant gB polypeptide having hyperfusogenic activity. The recombinant human VZV can contain a cell type specific promoter sequence that drives expression of an essential viral gene of the recombinant human VZV within the cancer cell. The recombinant human VZV can contain nucleic acid encoding a scFv fused to a VZV glycoprotein. The scFv can be capable of binding to a polypeptide expressed by the cancer cell. The recombinant human VZV can contain nucleic acid encoding a reporter polypeptide. The reporter polypeptide can be selected from the group consisting of hNIS and beta-hCG.

The second recombinant human herpesvirus can be a human beta herpesvirus. The human beta herpesvirus can be a recombinant human CMV. The recombinant human CMV can (a) have a mutation in the UL/b′ region, (b) have a mutation in the UL1-UL20 region, or (c) include nucleic acid encoding a functional pentameric gH/gL/UL128/UL 130/UL131 complex. For example, in some cases, the recombinant human CMV includes the mutation in the UL/b′ region. In some cases, the recombinant human CMV includes the mutation in the UL1-UL20 region. In some cases, the recombinant human CMV contains nucleic acid encoding the functional pentameric gH/gL/UL128/UL130/UL131 complex. The recombinant human CMV can contain nucleic acid encoding a retargeted Paramyxovirus fusogenic membrane glycoprotein (FMG) complex.

In another aspect, this document features a method for treating a mammal having cancer, where the method includes, or consists essentially of, administering, to the mammal, a composition provided herein. For example, the method can include administering to the mammal a composition containing, consisting essentially of, or consisting of a first recombinant human herpesvirus and a second recombinant human herpesvirus, where the first and second recombinant human herpesviruses are from two different herpesvirus sub-families, and where, when the composition is administered to a mammal having cancer, the first recombinant human herpesvirus infects a cancer cell and the second recombinant human herpesvirus infects a stromal cell associated with the cancer, thereby reducing the number of cancer cells within the mammal. The mammal can be a human. The method also can include administering, to the mammal, one or more therapies selected from the group consisting of radiation therapy, chemotherapy, and immunotherapy.

In another aspect, this document features a method for treating a mammal having cancer, where the method includes, consists essentially of, (a) administering, to the mammal, a first recombinant human herpesvirus, and (b) administering, to the mammal, a second recombinant human herpesvirus, where the first and second recombinant human herpesviruses are from two different herpesvirus sub-families, and where the first recombinant human herpesvirus infects a cancer cell of the mammal and the second recombinant human herpesvirus infects a stromal cell associated with the cancer, thereby reducing the number of cancer cells within the mammal. The mammal can be a human. The first recombinant human herpesvirus can be administered to the mammal before the second recombinant human herpesvirus. The first recombinant human herpesvirus can be administered to the mammal after the second recombinant human herpesvirus. The first recombinant human herpesvirus and the second recombinant human herpesvirus can be administered to the mammal at the same time. The method further can include administering, to the mammal, one or more therapies selected from the group consisting of radiation therapy, chemotherapy, and immunotherapy.

The first recombinant human herpesvirus can be a human alpha herpesvirus. The human alpha herpesvirus can be a recombinant human HSV or a recombinant human VZV. For example, the human alpha herpesvirus can be a recombinant human HSV. The recombinant human HSV can (a) lack both copies of ICP34.5, (b) lack functional ICP47, (c) contain nucleic acid encoding a mutant gB polypeptide having hyperfusogenic activity, or (d) contain a heterologous nucleic acid sequence encoding a therapeutic polypeptide. In some cases, the recombinant human HSV (a) lacks both copies of ICP34.5, or (b) lacks functional ICP47. In some cases, the recombinant human HSV lacks both copies of ICP34.5. In some cases, the recombinant human HSV lacks functional ICP47. The recombinant human HSV can contain nucleic acid encoding the mutant gB polypeptide. The recombinant human HSV can contain the heterologous nucleic acid sequence encoding a therapeutic polypeptide. The therapeutic polypeptide can be selected from the group consisting of pigment epithelium-derived factor (PEDF), CCL4, CXCL13, IL-12, IL-15, and brain-derived neurotropic factor (BDNF). The recombinant human HSV can (a) lack both copies of ICP34.5 or lacks functional ICP47, (b) contain nucleic acid encoding a mutant gB polypeptide having hyperfusogenic activity, and (c) contain a heterologous nucleic acid sequence encoding a therapeutic polypeptide (e.g., a therapeutic polypeptide is selected from the group consisting of PEDF, CCL4, CXCL13, IL-12, IL-15, and BDNF). The recombinant human HSV can contain a cell type specific promoter sequence that drives expression of an essential viral gene of the recombinant human HSV within the cancer cell. The recombinant human HSV can contain nucleic acid encoding a single-chain variable fragment (scFv) fused to an HSV glycoprotein. The scFv can be capable of binding to a polypeptide expressed by the cancer cell. The recombinant human HSV can contain nucleic acid encoding a reporter polypeptide. The reporter polypeptide can be selected from the group consisting of human sodium/iodide symporter (hNIS) and human chorionic gonadotropin beta subunit (beta-hCG).

The human alpha herpesvirus can be a recombinant human VZV. The recombinant human VZV can (a) be attenuated via natural genetic variability, an induced mutation, or reverse genetics or (b) contain a heterologous nucleic acid sequence encoding a therapeutic polypeptide. For example, in some cases, the recombinant human VZV is attenuated via natural genetic variability, an induced mutation, or reverse genetics. In some cases, the recombinant human VZV is attenuated via an induced mutation. In some cases, recombinant human VZV contains the heterologous nucleic acid sequence encoding a therapeutic polypeptide. The therapeutic polypeptide can be selected from the group consisting of PEDF, CCL4, CXCL13, IL-12, IL-15, and BDNF. The recombinant human VZV can contain nucleic acid encoding a mutant gB polypeptide having hyperfusogenic activity. The recombinant human VZV can contain a cell type specific promoter sequence that drives expression of an essential viral gene of the recombinant human VZV within the cancer cell. The recombinant human VZV can contain nucleic acid encoding a scFv fused to a VZV glycoprotein. The scFv can be capable of binding to a polypeptide expressed by the cancer cell. The recombinant human VZV can contain nucleic acid encoding a reporter polypeptide. The reporter polypeptide can be selected from the group consisting of hNIS and beta-hCG.

The second recombinant human herpesvirus can be a human beta herpesvirus. The human beta herpesvirus can be a recombinant human CMV. The recombinant human CMV can (a) have a mutation in the UL/b′ region, (b) have a mutation in the UL1-UL20 region, or (c) include nucleic acid encoding a functional pentameric gH/gL/UL128/UL130/UL131 complex. For example, in some cases, the recombinant human CMV includes the mutation in the UL/b′ region. In some cases, the recombinant human CMV includes the mutation in the UL1-UL20 region. In some cases, the recombinant human CMV contains nucleic acid encoding the functional pentameric gH/gL/UL128/UL130/UL131 complex. The recombinant human CMV can contain nucleic acid encoding a retargeted Paramyxovirus fusogenic membrane glycoprotein (FMG) complex.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 is a diagram depicting a herpesviral combination therapy according to some embodiments described herein.

FIG. 2 is a diagram depicting steps in a method for constructing of herpesviral BACs. The BAC vector containing a left homology arm and a right homology arm was linearized and transfected into Vero cells, MRC-5 cells, NIH-3T3 cells, or ARPE-19 cells. The cells contained herpesviral linear genome or replicating viruses to generate the recombinant herpesviral BAC viruses. Green or red plaques of the recombinant virus were purified, and circular viral DNA was isolated from the BAC virus infected cells. The circular viral BAC DNA was transformed into E. coli DH10B cells, and colonies containing full length herpesviral genome were selected. The BAC plasmid carrying the viral genome was extracted and transferred into E. coli SW102 cells containing a lambda red recombineering system. The large herpesviral BAC plasmid can be genetically manipulated within the SW102 cells, and the BAC sequence can be removed by transfecting the BAC containing plasmid into Vero cells, MRC-5 cells, NIH-3T3 cells, or ARPE-19 cells that stably express Cre recombinase.

FIGS. 3A-3F depict construction of HSV-1 BAC recombinants KOS-BAC-NIS-hCG (FIG. 3A), KOS-BAC-NIS-hCG-ΔICP34.5 (FIG. 3B), KOS-BAC-NIS-hCG-gBsyn-ΔICP34.5 (FIG. 3C), KOS-BAC-NIS-hCG-gBsyn-ΔICP34.5-IL12-PEDF (FIG. 3D), KOS-BAC-NIS-hCG-gBsyn-ΔICP34.5-IL12-IL15RAIL15 (FIG. 3E), and KOS-BAC-NIS-hCG-gBsyn-ΔICP34.5-IL12-IL15RAIL 15-PEDF (FIG. 3F).

FIGS. 4A-4E depict construction of CMV BAC recombinants HCMV AD169r-BAC: gH/gL/UL128-UL131 repaired (FIG. 4A), HCMV AD16-BAC-AUS1-US11 (FIG. 4B), HCMV AD169r-BAC-CD::UPRT (FIG. 4C), HCMV AD169r-BAC-H/F (FIG. 4D), and HCMV AD169r-BAC-HaaEGFR/F (FIG. 4E).

FIG. 5 shows steps for constructing VZV BAC recombinants vOka-BAC, vOka-BAC bHCG, and vOka-BAC bHCG-gBsyn (gB^{Y881F}).

FIGS. 6A-6H show results from in vitro characterization of virus growth and reporter expression. FIGS. 6A and 6B are graphs plotting growth curves for HSV-1 KOS-BACs. Vero cells were infected with KOS-BACs at MOI=0.01, and virus titers from supernatants for each group were determined by plaque assay at the indicated time points. Results are shown with both linear (FIG. 6A) and Log10 (FIG. 6B) Y-axes. FIGS. 6C-6E are graphs plotting expression of NIS (FIGS. 6C and 6D) and beta-hCG (FIG. 6E). Vero cells were infected with KOS-BACs at MOI-0.01, and NIS expression on attached cells and beta-hCG released into cell supernatants for each group were determined by I¹²⁵ uptake assay and ELISA, respectively. The I¹²⁵ uptake results are shown with both linear (FIG. 6C) and Log10 (FIG. 6D) Y-axes. CPM, counts per minute. FIGS. 6F and 6G are graphs plotting growth curves of HCMV AD169-BACs. MOI=0.01 in MRC-5 cells, no fusion inhibition peptide added. Titers: Cell free plus cell associated. FIG. 6H is a graph plotting peak titers of vOka-bHCG and v-Oka-bHCG-gBsyn. MOI=0.0001 in ARPE-19 cells.

FIGS. 7A-7E show the results of using a HSV-1 plus mCMV combination in A20 B cell lymphoma and EMT6 breast cancer syngeneic models. Tumor cells were implanted on both flanks subcutaneously at day 0. When average tumor size reached a diameter of 5 mm, 50 μL of PBS, or HSV KOS-BAC-NIS-bHCG-ΔICP47 (1×10⁶ pfu per injection), or a combination of HSV KOS-BAC-NIS-bHCG-ΔICP47 (1×10⁶ pfu per injection) plus mCMV smith (1×10⁵ pfu per injection), or mCMV smith (1×10⁵ pfu per injection) were intratumorally injected into the tumors on right flank (totally 3 injections per mouse, every other day). FIG. 7A depicts the study protocol and includes a graph plotting animal survival in the A20 model. FIG. 7B depicts the study protocol and includes a graph plotting animal survival in the EMT6 model. FIGS. 7C and 7D are graphs plotting beta-hCG levels in tumors (FIG. 7C) and blood (FIG. 7D). FIG. 7E is a graph plotting the amount of virus recovered from the indicated tissues.

FIG. 8 is a series of images showing the results of immunofluorescence (IFA) analysis after intratumoral co-infection with HSV-1 and mCMV. Co-staining of HSV (GFP) and mCMV (gB) were performed in EMT6 tumors collected at day 3 post first co-injection. Macrophage markers F4/80 and CD11b were co-stained. HSV-1 and mCMV targeted different cell types within the EMT6 tumors, and mCMV also targeted tumor-associated macrophages. Top row: DAPI, GFP, gB, and F4/80 co-staining; bottom row: DAPI, GFP, gB, and CD11b co-staining, plus merged results at the left of each row. The top row shows mCMV gB and F4/80 co-localization, while the bottom shows co-localization of mCMV gB and CD11b.

FIGS. 9A-9I show upregulation of cytokines in HSV plus mCMV co-injected EMT6 tumors. FIG. 9A is a graph plotting volumes for tumors infected with HSV, mCMV, HSV plus mCMV, or PBS. FIG. 9B shows cytokine profiling with antibody array for 3 dpi tumors. FIG. 9C shows cytokine profiling with antibody array for 9 dpi tumors. FIGS. 9D-9I are graphs plotting the results of ELISA analysis of cytokines in injected tumors. FIG. 9D, PEDF; FIG. 9E, VEGF; FIG. 9F, CCL4; FIG. 9G, CSCL13; FIG. 9H, IL12 p70; and FIG. 9I, IL15.

FIGS. 10A-10K show a study protocol (FIG. 10A) and results using HSV-1 variants plus mCMV in EMT6 model groups. Tumor cells were implanted in both flanks subcutaneously at day 0. When the average tumor size reached diameter of 5 mm, 50 μL of PBS, or a combination of a HSV KOS-BAC-NIS-bHCG variant (1×10⁶ pfu per injection) plus mCMV Smith (1×10⁵ pfu per injection), were intratumorally injected into the tumors on the right flank (a total of 3 injections per mouse, every other day). Tumor volumes, animal survival and blood beta-hCG levels were monitored and analyzed. FIG. 10B, tumor volume with PBS or WT plus mCMV; FIG. 10C, tumor volume with PBS or dICP47 plus mCMV; FIG. 10D, tumor volume with PBS or dICP34.5 plus mCMV; FIG. 10E, tumor volume with PBS or dICP0 plus mCMV; FIG. 10F, tumor volume with PBS or dICP27 plus mCMV; FIG. 10G, tumor volume with PBS or UL41 plus mCMV; FIG. 10H, tumor volume with PBS or dICP6 plus mCMV; FIG. 10I, tumor volume with PBS or gB R858H plus mCMV; FIG. 10J, mouse survival; and FIG. 10K, blood beta-hCG.

FIG. 11 includes a diagram of a KOS-BAC-NIS-hCG-gBsyn-Δ/CP34.5-Transgene construct (top), and images showing that viruses containing the indicated constructs were reconstituted in Vero-cre cells (bottom).

FIGS. 12A-12H shows a study protocol (FIG. 12A) and results using KOS-BAC-NIS-hCG-gBsyn-Δ/CP34.5-Transgene plus mCMV in the EMT6 model. Tumor cells were implanted in both flanks subcutaneously at day 0. When the average tumor size reached a diameter of 5 mm, 50 μL of PBS, or a combination of a HSV KOS-BAC-NIS-bHCG variant (1×10⁶ pfu per injection) plus mCMV smith (1×10⁵ pfu per injection), were intratumorally injected into the tumors on the right flank (a total of 3 injections per mouse, every other day). Tumor volumes were monitored: FIG. 12B, PBS or Vector plus mCMV; FIG. 12C, PBS or PEDF plus mCMV; FIG. 12D, PBS or CCL4 plus mCMV; FIG. 12E, PBS or CXCL13 plus mCMV; FIG. 12F, PBS or IL12 plus mCMV; FIG. 12G, PBS or IL15RAsushiIL 15 plus mCMV; and FIG. 12H, crBDNF plus mCMV. CR, complete remission.

FIGS. 13A-13C are graphs plotting body weights (FIG. 13A), blood beta-hCG levels (FIG. 13B), and survival (FIG. 13C) for EMT6 model mice injected with KOS-BAC-NIS-hCG-gBsyn-Δ/CP34.5-Transgene plus mCMV constructs. Tumor cells were implanted in both flanks subcutaneously at day 0. When the average tumor size reached a diameter of 5 mm, 50 μL of PBS, or a combination of HSV KOS-BAC-NIS-bHCG variant (1×10⁶ pfu per injection) plus mCMV smith (1×10⁵ pfu per injection), were intratumorally injected into the tumors on the right flank (a total of 3 injections per mouse, every other day).

FIGS. 14A and 14B are graphs plotting results using VZV in a MeWo melanoma model. MeWo cells were implanted subcutaneously into the right flank of nude mice (5×10⁶ cells/mouse) at day 0, and at days 13, 15, and 17, thawed Vero cells or v-Oka infected Vero cells (1.5×10⁵ pfu total injected per mouse) were IT injected into the MeWo tumors. Tumor volume was monitored (FIG. 14A). Blood samples were collected at day post first virus injection, and blood beta-hCG levels were measured (FIG. 14B).

FIGS. 15A-15D show results obtained after delivering HCMV infected macrophages into solid tumors. FIG. 15A is a graph plotting in vitro cytotoxicity of HCMV Merlin and AD169r-BAC to monocyte-derived macrophages. FIG. 15B shows the effects of HCMV infection on macrophage cytokine secretion, as assessed by cytokine profiling with antibody array. Monocyte derived macrophages were infected with HCMV at MOI-0.6. FIG. 15C is a graph plotting tumor volumes in the mice after injection of HCMV infected macrophages into U251 tumors. U251 cells were subcutaneously implanted on the right flanks of nude mice at day 0, and at days 13, 15, and 17, macrophages exposed to virus (MOI-0.6, 2 hours prior to injection) were intratumorally injected into tumors. FIG. 15D is a graph plotting survival of the animals.

FIGS. 16A-16B. HSV KOS-BAC-NIS-hCG-gBsyn-ΔICP34.5-IL12+HCMV Merlin in EMT6 model groups. Tumor cells were implanted on both flanks subcutaneously at day 0. When average tumor size reached diameter of 5 mm (on day 8), 50 μL of PBS, or co-injection of KOS-BAC-NIS-hCG-gBsyn-ΔICP34.5-IL12 (1×10⁶ pfu per injection)+HCMV Merlin (1×10⁵ pfu per injection), were intratumorally injected into the tumors on the right flank (in total: 3 injections per mouse, every other day). 20 Tumor volumes (FIG. 16A) and animal survival (FIG. 16B) were monitored and analyzed.

### DETAILED DESCRIPTION

Cancer cells are abnormal cells that divide without control, invade nearby tissues, form solid tumors or flood the circulation, and under certain circumstances establish metastasis. Cancer associated stromal cells are cell types within a tumor that surround and support the cancerous cells. Stromal cells include, for example, tumor infiltrated lymphocytes and myeloid cells, endothelial cells, fibroblasts, adipocytes, and stellate cells, and their phenotypes can be shifted from tumor-suppressive to tumor promoting, such that they can promote growth, invasion, and metastasis.

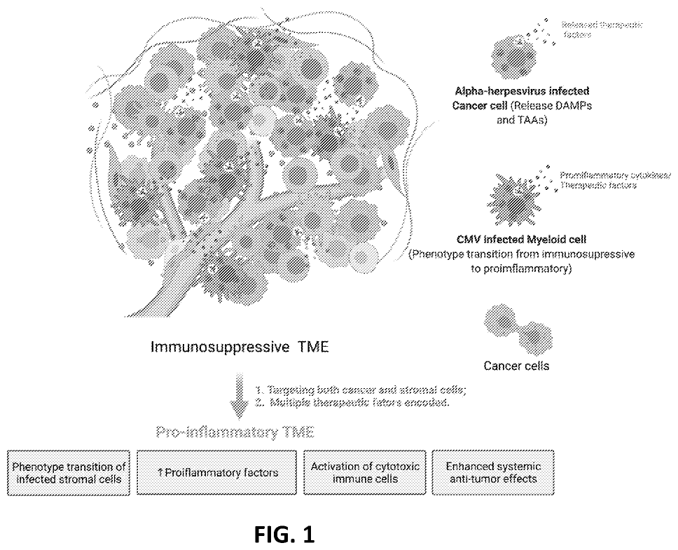

The methods and materials described herein provide herpesvirus combination therapies in which two or more herpesviruses are combined for treating diseases such as cancer. As described herein, for example, an alpha herpesvirus plus beta herpesvirus combination therapeutic platform has been developed, with an epithelial tropic alpha herpesvirus (e.g., HSV or VZV) to target cancer cells and trigger innate/adaptive anti-tumor responses, and a macrophage/endothelial tropic beta herpesvirus (e.g., CMV) to target cancer-associated macrophages and endothelial cells in order to reverse or minimize the immune-suppressive environment (FIG. 1). The herpesviruses also can be used to deliver therapeutic transgenes with efficacy, when expressed, to treat disease (e.g., cancer). In some cases, for example, the expression of a therapeutic transgene can enhance an anti-tumor immune-response. Transgenes can be transferred naturally or by genetic engineering into a distinct vector or host for subsequent delivery into a host.

In some cases, a herpesvirus construct provided herein also can contain one or more nucleic acid sequences that encode a reporter gene to enable detection of vector replication levels and/or location, and/or one or more cell type specific promoters to drive expression of a coding sequence (e.g., a transgene sequence) in particular cell types. In some cases, a herpesvirus can encode a single-chain variable fragment (scFv) that can bind to a polypeptide expressed by a cancer cell. scFv polypeptides are fusion polypeptides that contain the heavy and light chain variable regions of an immunoglobulin and a short linker peptide to connect the variable regions. An scFv can retain the binding specificity of the original immunoglobulin, and can be produced directly from cell cultures. In some cases, a herpesvirus provided herein can encode a retargeted Paramyxovirus fusogenic membrane glycoprotein (FMG) complex. Paramyxovirus FMG is a component of the viral envelope that recognizes specific cell receptors and mediates extracellular viral envelope to cell membrane fusion, as well as intracellular fusion between an infected cell and neighbor cells. A retargeted Paramyxovirus FMG can be a recombinant fusogenic viral glycoprotein with impaired endogenous receptor binding ability, and an scFV domain fused in frame to endow the recombinant glycoprotein with retargeted binding specificity.

This document provides methods and materials for treating cancer. For example, this document provides methods and materials for treating cancer using two or more herpesviruses from different sub-families as oncolytic agents. In some cases, this document provides combinations of recombinant herpesviruses from different sub-families (e.g., alpha and beta sub-families) having oncolytic anti-cancer activity. In some cases, this document provides methods for using a combination of two or more recombinant herpesviruses provided herein to treat a mammal having, or at risk of having, cancer. For example, two or more recombinant herpesviruses from different sub-families can be administered to a mammal having, or at risk of having, cancer, to reduce the number of cancer cells and cancer-associated stromal cells (e.g., by infecting and killing cancer cells and cancer-associated stromal cells) in the mammal (e.g., a human). For example, two or more recombinant herpesviruses can be administered to a mammal having, or at risk of having, cancer, to stimulate anti-cancer immune responses in the mammal (e.g., a human). In some cases, a HSV and a CMV (e.g., a HCMV or a mCMV) can be used in combination to treat a mammal having, or at risk of having, cancer. In some cases, a VZV and a CMV (e.g., a HCMV or a mCMV) can be used in combination to treat a mammal having, or at risk of having, cancer.

Recombinant herpesviruses described herein can include one or more nucleotide sequences that do not naturally occur in that herpesvirus genome (e.g., do no naturally occur in that herpesvirus prior to recombination). Nucleotide sequences that do not naturally occur in the herpesvirus can be from any appropriate source. In some cases, a nucleotide sequence that does not naturally occur in that herpesvirus can be from a non-viral organism. In some cases, a nucleotide sequence that does not naturally occur in that herpesvirus can be from a virus other than a herpesvirus. In some cases, a nucleotide sequence that does not naturally occur in that herpesvirus can be a synthetic nucleotide sequence.

In some cases, a recombinant herpesvirus described herein (e.g., a recombinant HSV, VZV, or CMV having oncolytic anti-cancer activity) can include a herpesvirus genome containing one or more modifications to one or more nucleic acids encoding a polypeptide and/or one or more viral elements of the herpesvirus genome. The one or more modifications can be any appropriate modification. Examples of modifications that can be made to a nucleic acid encoding a polypeptide or to a viral element include, without limitation, deletions, insertions, and substitutions.

For example, a recombinant herpesvirus can have one or more genomic deletions. In some cases, a recombinant HSV can have a deletion (e.g., a full deletion or a partial deletion) of the ICP47 nucleic acid. In some cases, a recombinant HSV can have a deletion (e.g., a full deletion or a partial deletion) of the ICP34.5s nucleic acid. In some cases, a recombinant mCMV can have a deletion (e.g., a full deletion or a partial deletion) such that it lacks a functional homolog of HCMV UL128-131. In some cases, a recombinant CMV can have a mutation or a deletion (e.g., a full deletion or a partial deletion) in the UL/b′ region such that it lacks a functional homolog of HCMV UL/b′. In some cases, a recombinant CMV can have a mutation or a deletion (e.g., a full deletion or a partial deletion) in the UL1-UL20 region such that it lacks a functional homolog of HCMV UL1-UL20. In some cases, a recombinant HCMV can have a deletion of the US1-US11 region.

For example, a recombinant herpesvirus can have one or more substitutions. In some cases, a recombinant HSV can have a mutation in the gBsyn nucleic acid (e.g., a mutation that results in a R858H substitution in the encoded polypeptide), such that the HSV includes the mutated gBsyn nucleic acid and expresses the encoded gBsyn polypeptide. In some cases, a recombinant HSV or a recombinant VZV can have a mutation in the gB nucleic acid (e.g., a gB hyperfusogenic mutation resulting in a Y881F substitution in the encoded polypeptide), such that the HSV or VZV includes the mutated gB nucleic acid and expresses the encoded gB polypeptide. A representative example of a nucleotide sequence encoding a HSV-1 hyperfusogenic gB polypeptide is set forth in SEQ ID NO:1, and the amino acid sequence of the encoded HSV-1 hyperfusogenic polypeptide is set forth in SEQ ID NO:2 (both shown in TABLE 1; the location of the R858H mutation is indicated by underlining in SEQ ID NO:2). A representative example of a nucleotide sequence encoding a VZV hyperfusogenic gB polypeptide is set forth in SEQ ID NO:3, and the amino acid sequence of the encoded VZV hyperfusogenic polypeptide is set forth in SEQ ID NO:4 (both shown in TABLE 1; the location of the Y881F mutation is indicated by underlining in SEQ ID NO:4).

For example, a recombinant herpesvirus can include one or more genomic insertions (e.g., insertion of one or more transgenes). In some cases, a recombinant herpesvirus can include a transgene (e.g., a nucleic acid encoding a suicide polypeptide), and can express the encoded polypeptide. In some cases, a recombinant herpesvirus can include a regulatory element (e.g., promoter such as a cell type-specific promoter). For example, a recombinant herpesvirus can include one or more genomic substitutions (e.g., a substitution of one or more nucleic acids encoding a polypeptide with one or more transgenes).

In cases where a recombinant herpesvirus described herein (e.g., a recombinant HSV, VZV, or CMV) includes a transgene, the transgene can be any appropriate transgene. In some cases, a transgene can be a nucleotide sequence encoding a reporter. Examples of reporters include, without limitation, human sodium/iodide symporter (hNIS) and human chorionic gonadotropin beta subunit (beta-hCG).

For example, a recombinant herpesvirus provided herein can include a hNIS nucleotide sequence and can express the encoded hNIS polypeptide. Any appropriate nucleic acid encoding a NIS polypeptide can be inserted into a herpesvirus genome. For example, nucleic acid encoding a human NIS polypeptide can be inserted into the genome of HSV or VZV. A representative example of a nucleotide sequence encoding a human NIS polypeptide is set forth in SEQ ID NO:5, and the amino acid sequence of the encoded human NIS polypeptide is set forth in SEQ ID NO:6 (both shown in TABLE 1).

For example, a recombinant herpesvirus provided herein can include a beta-hCG nucleotide sequence and can express the encoded beta-hCG polypeptide. A representative example of a nucleotide sequence encoding a beta-hCG polypeptide is set forth in SEQ ID NO: 7, and the amino acid sequence of the encoded beta-hCG polypeptide is set forth in SEQ ID NO:8 (both shown in TABLE 1). In some cases, a recombinant herpesvirus provided herein can include a transgene encoding a detectable label, and can express the detectable label. Examples of detectable labels include, without limitation, fluorophores (e.g., green fluorescent protein (GFP), mCherry, yellow fluorescent protein (YFP), cyan fluorescent protein (CFP), and Tomato), enzymes (e.g., luciferase, CRISPR associated protein 9 (Cas9), Cre recombinase, restriction enzymes, convertases, and thymidine kinases), and antigens.

In some cases, a transgene can be a nucleotide sequence encoding another useful polypeptide. Examples of other useful polypeptides include, without limitation, targeting polypeptides (e.g., ligands (e.g., natural ligands and artificial ligands) for cell surface receptors such as cytokines and hormones, single chain antibodies (e.g., targeting cancer antigens such as HER2, CA125, CD19, CD30, CD33, CD123, FLT3, BCMA, CEA, melan-A antigen, and PSA), and other viral envelopes), transport polypeptides (e.g., nuclear localization sequences (NLSs), mitochondrion targeting sequences, and lysosome targeting sequences), therapeutic polypeptides (e.g., immunomodulatory factors such as chemokines and cytokines, antibodies such as antibodies blocking immune checkpoint molecules (e.g., PD-1, PDL-1, and CTLA-4), genome editing systems, viral polypeptides, and gene repair polypeptides), and cytotoxic polypeptides (e.g., suicide polypeptides such as thymidine kinases, inducible Caspase 9 (iCasp9), viral polypeptides, and nitroreductase).

In some cases, for example, a recombinant herpesvirus (e.g., a recombinant HSV) can include a nucleic acid encoding a CXCL-13 polypeptide, and can express the encoded CXCL-13 polypeptide. In some cases, for example, a recombinant VZV can include a nucleic acid encoding a CXCL-13 polypeptide and can express the encoded CXCL-13 polypeptide. A representative example of a nucleotide sequence encoding a CXCL-13 polypeptide is set forth in SEQ ID NO:9, and the amino acid sequence of the encoded CXCL-13 polypeptide is set forth in SEQ ID NO:10 (both shown in TABLE 1).

In some cases, a recombinant herpesvirus (e.g., a recombinant HSV) can include a nucleic acid encoding a CCL-4 polypeptide, and can express the encoded CCL-4 polypeptide. In some cases, a recombinant VZV can include a nucleic acid encoding a CCL-4 polypeptide and can express the encoded CCL-4 polypeptide. A representative example of a nucleotide sequence encoding a CCL-4 polypeptide is set forth in SEQ ID NO: 11, and the amino acid sequence of the encoded CCL-4 polypeptide is set forth in SEQ ID NO: 12 (both shown in TABLE 1).

In some cases, a recombinant herpesvirus (e.g., a recombinant HSV) can include a nucleic acid encoding a PEDF polypeptide, and can express the encoded PEDF polypeptide. In some cases, a recombinant VZV can include a nucleic acid encoding a PEDF polypeptide and can express the encoded PEDF polypeptide. A representative example of a nucleotide sequence encoding a PEDF polypeptide is set forth in SEQ ID NO: 13, and the amino acid sequence of the encoded PEDF polypeptide is set forth in SEQ ID NO:14 (both shown in TABLE 1).

In some cases, a recombinant herpesvirus (e.g., a recombinant HSV) can include a nucleic acid encoding an IL 12 polypeptide, and can express the encoded IL-12 polypeptide. In some cases, a recombinant VZV can include a nucleic acid encoding an IL12 polypeptide and can express the encoded IL-12 polypeptide. A representative example of a nucleotide sequence encoding a IL-12 polypeptide is set forth in SEQ ID NO: 15, and the amino acid sequence of the encoded IL-12 polypeptide is set forth in SEQ ID NO: 16 (both shown in TABLE 1).

In some cases, a recombinant herpesvirus (e.g., a recombinant HSV) can include a nucleic acid encoding an IL 15RAsushi-IL15 polypeptide, and can express the encoded IL15RAsushi-IL 15 polypeptide. In some cases, a recombinant VZV can include a nucleic acid encoding an IL15RAsushi-IL15 polypeptide and can express the encoded IL15RAsushi-IL15 polypeptide. A representative example of a nucleotide sequence encoding an IL15RAsushi-IL15 polypeptide is set forth in SEQ ID NO:17, and the amino acid sequence of the encoded IL 15RAsushi-IL 15 polypeptide is set forth in SEQ ID NO: 18 (both shown in TABLE 1).

In some cases, a recombinant herpesvirus (e.g., a recombinant HSV) can include a nucleic acid encoding a mutant (e.g., cleavage-resistant) brain derived neurotropic factor (mutBDNF) polypeptide, and can express the encoded mutBDNF polypeptide. In some cases, a recombinant VZV can include a nucleic acid encoding a mutBDNF polypeptide and can express the encoded mutBDNF polypeptide. A representative example of a nucleotide sequence encoding a cleavage resistant BDNF polypeptide is set forth in SEQ ID NO: 19, and the amino acid sequence of the encoded cleavage resistant BDNF polypeptide is set forth in SEQ ID NO:20 (both shown in TABLE 1).

In some cases, a recombinant herpesvirus (e.g., a recombinant HCMV lacking a functional UL131 coding sequence) can include a nucleic acid encoding a functional UL131 polypeptide, where the transgene is inserted to replace the non-functional UL131 sequence, and where the recombinant herpesvirus can express the functional UL131 polypeptide. A representative example of a nucleotide sequence encoding a UL131 polypeptide is set forth in SEQ ID NO:21, and the amino acid sequence of the encoded UL131 polypeptide is set forth in SEQ ID NO:22 (both shown in TABLE 1).

In some cases, a recombinant herpesvirus (e.g., a recombinant HCMV) can include a nucleic acid encoding a CD::UPRT fusion suicide protein and can express the encoded CD::UPRT fusion. A representative example of a nucleotide sequence encoding a CD::UPRT fusion polypeptide is set forth in SEQ ID NO:23, and the amino acid sequence of the encoded CD::UPRT fusion polypeptide is set forth in SEQ ID NO:24 (both shown in TABLE 1).

In some cases, a recombinant herpesvirus (e.g., a recombinant HCMV) can include a nucleic acid encoding components of a Paramyxovirus H/Fs complex, and can express the encoded components of the H/Fs complex. A representative example of a nucleotide sequence encoding a Paramyxovirus H polypeptide is set forth in SEQ ID NO: 25, and the amino acid sequence of the encoded Paramyxovirus H polypeptide is set forth in SEQ ID NO:26 (both shown in TABLE 1). A representative example of a nucleotide sequence encoding a Paramyxovirus F polypeptide is set forth in SEQ ID NO: 27, and the amino acid sequence of the encoded Paramyxovirus F polypeptide is set forth in SEQ ID NO:28 (both shown in TABLE 1).

In some cases, a recombinant herpesvirus (e.g., a recombinant HCMV) can include a nucleic acid encoding an EGFR retargeted Paramyxovirus H polypeptide, and can express the encoded EGFR retargeted Paramyxovirus H polypeptide. A representative example of a nucleotide sequence encoding an EGFR retargeted Paramyxovirus H polypeptide is set forth in SEQ ID NO:29, and the amino acid sequence of the encoded EGFR retargeted Paramyxovirus H polypeptide is set forth in SEQ ID NO: 30 (both shown in TABLE 1).

In some cases, a recombinant herpesvirus can include a nucleic acid encoding a scFv polypeptide targeting EGFR, HER2, vascular endothelial growth factor receptor (VEGF), or CD19, and can express the encoded scFv polypeptide targeting EGFR, HER2, vascular endothelial growth factor receptor (VEGF), or CD19.

In some cases, a recombinant herpesvirus (e.g., a recombinant HCMV) can include a nucleic acid encoding a functional pentameric gH/gL/UL128/UL130/UL131 complex, and can express the functional pentameric gH/gL/UL128/UL130/UL131 complex.

TABLE 1
Representative sequences

HSV-1 hyperfusogenic gB DNA sequence:
atgcaccagggcgccccctcgtgggggcgccggtggttcgtcgtatgggcgctcttggggttgacgctgggggtcctggtggc
gtcggcggctccgagttcccccggcacgcctggggtcgcggccgcgacccaggcggcgaacgggggccctgccactccgg
cgccgcccgcccttggcgccgccccaacgggggacccgaaaccgaagaagaacaaaaaaccgaaaaacccaacgccacc
acgccccgccggcgacaacgcgaccgtcgccgcgggccacgccaccctgcgcgagcacctgcgggacatcaaggcggag
aacaccgatgcaaacttttacgtgtgcccaccccccacgggcgccacggtggtgcagttcgagcagccgcgccgctgcccga
cccggcccgagggtcagaactacacggagggcatcgcggtggtcttcaaggagaacatcgccccgtacaagttcaaggccac
catgtactacaaagacgtcaccgtttcgcaggtgtggttcggccaccgctactcccagtttatggggatctttgaggaccgcgccc
ccgtccccttcgaggaggtgatcgacaagatcaacgccaagggggtctgtcggtccacggccaagtacgtgcgcaacaacct
ggagaccaccgcgtttcaccgggacgaccacgagaccgacatggagctgaaaccggccaacgccgcgacccgcacgagc
cggggctggcacaccaccgacctcaagtacaacccctcgcgggtggaggcgttccaccggtacgggacgacggtaaactgc
atcgtcgaggaggtggacgcgcgctcggtgtacccgtacgacgagtttgtgctggcgactggcgactttgtgtacatgtccccgt
tttacggctaccgggaggggtcgcacaccgaacacaccagctacgccgccgaccgcttcaagcaggtcgacggcttctacgc
gcgcgacctcaccaccaaggcccgggccacggcgccgaccacccggaacctgctcacgacccccaagttcaccgtggcctg
ggactgggtgccaaagcgcccgtcggtctgcaccatgaccaagtggcaggaagtggacgagatgctgcgctccgagtacgg
cggctccttccgattctcctccgacgccatatccaccaccttcaccaccaacctgaccgagtacccgctctcgcgcgtggacctg
ggggactgcatcggcaaggacgcccgcgacgccatggaccgcatcttcgcccgcaggtacaacgcgacgcacatcaaggtg
ggccagccgcagtactacctggccaatgggggctttctgatcgcgtaccagccccttctcagcaacacgctcgcggagctgtac
gtggggaacacctccgagagcagagccgcaagcccccaaaccccacgcccccgccgcccggggccagcgccaacgcgt
ccgtggagcgcatcaagaccacctcctccatcgagttcgcccggctgcagtttacgtacaaccacatacagcgccatgtcaacg
atatgttgggccgcgttgccatcgcgtggtgcgagctacagaatcacgagctgaccctgtggaacgaggcccgcaagctgaac
cccaacgccatcgcctcggtcaccgtgggccggcgggtgagcgcgcggatgctcggcgacgtgatggccgtctccacgtgc
gtgccggtcgccgcggacaacgtgatcgtccaaaactcgatgcgcatcagctcgcggcccggggcctgctacagccgccccc
tggtcagctttcggtacgaagaccagggcccgttggtcgaggggcagctgggggagaacaacgagctgcggctgacgcgcg
atgcgatcgagccgtgcaccgtgggacaccggcgctacttcaccttcggtgggggctacgtgtacttcgaggagtacgcgtact
cccaccagctgagccgcgccgacatcaccaccgtcagcaccttcatcgacctcaacatcaccatgctggaggatcacgagtttg
tccccctggaggtgtacacccgccacgagatcaaggacagcggcctgctggactacacggaggtccagcgccgcaaccagc
tgcacgacctgcgcttcgccgacatcgacacggtcatccacgccgacgccaacgccgccatgttcgcgggcctgggcgcgtt
cttcgaggggatgggcgacctggggcgcgcggtcggcaaggtggtgatgggcatcgtgggcggcgtggtatcggccgtgtc
gggcgtgtcctccttcatgtccaacccctttggggcgctggccgtgggtctgttggtcctggccggcctggcggcggccttcttc
gcctttcgttacgtcatgcggctgcagagcaaccccatgaaggccctgtaccctctaaccaccaaggagctcaagaaccccacc
aacccggacgcgtccggggagggcgaggagggcggcgactttgacgaggccaagctagccgaggccagggagatgatac
ggtacatggccctggtgtcggccatggagcacacggaacacaaggccaagaagaagggcacgagcgcgctgctcagcgcc
aaggtcaccgacatggtcatgcgcaagcgccgcaacaccaactacacccaagttcccaacaaagacggtgacgccgacgag
gacgacctgtga (SEQ ID NO: 1)
HSV-1 hyperfusogenic gB amino acid sequence:
MHQGAPSWGRRWFVVWALLGLTLGVLVASAAPSSPGTPGVAAATQAANGGPA
TPAPPALGAAPTGDPKPKKNKKPKNPTPPRPAGDNATVAAGHATLREHLRDIKA
ENTDANFYVCPPPTGATVVQFEQPRRCPTRPEGQNYTEGIAVVFKENIAPYKFKA
TMYYKDVTVSQVWFGHRYSQFMGIFEDRAPVPFEEVIDKINAKGVCRSTAKYVR
NNLETTAFHRDDHETDMELKPANAATRTSRGWHTTDLKYNPSRVEAFHRYGTT
VNCIVEEVDARSVYPYDEFVLATGDFVYMSPFYGYREGSHTEHTSYAADRFKQV
DGFYARDLTTKARATAPTTRNLLTTPKFTVAWDWVPKRPSVCTMTKWQEVDE
MLRSEYGGSFRFSSDAISTTFTTNLTEYPLSRVDLGDCIGKDARDAMDRIFARRY
NATHIKVGQPQYYLANGGFLIAYQPLLSNTLAELYVREHLREQSRKPPNPTPPPP
GASANASVERIKTTSSIEFARLQFTYNHIQRHVNDMLGRVAIAWCELQNHELTL
WNEARKLNPNAIASVTVGRRVSARMLGDVMAVSTCVPVAADNVIVQNSMRISS
RPGACYSRPLVSFRYEDQGPLVEGQLGENNELRLTRDAIEPCTVGHRRYFTFGGG
YVYFEEYAYSHQLSRADITTVSTFIDLNITMLEDHEFVPLEVYTRHEIKDSGLLDY
TEVQRRNQLHDLRFADIDTVIHADANAAMFAGLGAFFEGMGDLGRAVGKVVM
GIVGGVVSAVSGVSSFMSNPFGALAVGLLVLAGLAAAFFAFRYVMRLQSNPMK
ALYPLTTKELKNPTNPDASGEGEEGGDFDEAKLAEAREMIRYMALVSAMEHTEH
KAKKKGTSALLSAKVTDMVMRKRRNTNYTQVPNKDGDADEDDL (SEQ ID
NO: 2)
VZV hyperfusogenic gB DNA sequence:
atgtccccttgtggctattattcaaagtggagaaacagggatcgaccagaataccgtcgtaatctacgattcagacgttttttctcttc
tatacaccctaatgcagcggctggctccggattcaacggacccggcgttttcataacctccgttacgggggtgtggttatgcttttt
atgcatattttctatgtttgttacggcggttgtgtcggtctctccaagctcgttttatgagagtttacaagtagagcccacacaatcaga
agatataacccggtctgctcatctgggcgatggtgatgaaatcagagaagctatacacaagtcccaggacgccgaaacaaaac
ccacgttttacgtctgcccaccgccaacaggctccacaatcgtacgattagaaccacctcggacatgtccggattatcaccttggt
aaaaactttacagagggtattgctgttgtttataaagaaaacattgccgcgtacaagtttaaggcgacggtatattacaaagatgtta
tcgttagcacggcgtgggccggaagttcttatacgcaaattactaatagatatgctgatagggtaccaattcccgtttcagagatca
cggacaccattgataagtttggcaagtgttcttctaaagcaacgtacgtacgaaataaccacaaagttgaagcctttaatgaggata
aaaatccacaggatatgcctctaatcgcatcaaaatataattctgtgggatccaaagcatggcatactaccaatgacacgtacatg
gttgccggaacccccggaacatataggacgggcacgtcggtgaattgcatcattgaggaagttgaagccagatcaatattccctt
atgatagttttggactttccacgggagatataatatacatgtccccgttttttggcctacgggatggtgcatacagagaacattccaat
tatgcaatggatcgttttcaccagtttgagggttatagacaaagggatcttgacactagagcattactggaacctgcagcgcggaa
ctttttagtcacgcctcatttaacggttggttggaactggaagccaaaacgaacggaagtttgttcgcttgtcaagtggcgtgaggtt
gaagacgtagttcgcgatgagtatgcacacaattttcgctttacaatgaaaacactttctaccacgtttataagtgaaacaaacgagt
ttaatcttaaccaaatccatctcagtcaatgtgtaaaggaggaagcccgggctattattaaccggatctatacaaccagatacaact
catctcatgttagaaccggggatatccagacctaccttgccagaggggggtttgttgtggtgtttcaacccctgctgagcaattccc
tcgcccgtctctatctccaagaattggtccgtgaaaacactaatcattcaccacaaaaacacccgactcgaaataccagatcccga
cgaagcgtgccagttgagttgcgtgccaatagaacaataacaaccacctcatcggtggaatttgctatgctccagtttacatatga
ccacattcaagagcatgttaatgaaatgttggcacgtatctcctcgtcgtggtgccagctacaaaatcgcgaacgcgccctttgga
gcggactatttccaattaacccaagtgctttagcgagcaccattttggatcaacgtgttaaagctcgtattctcggcgacgttatctc
cgtttctaattgtccagaactgggatcagatacacgcattatacttcaaaactctatgagggtatctggtagtactacgcgttgttata
gccgtcctttaatttcaatagttagtttaaatgggtccgggacggtggagggccagcttggaacagataacgagttaattatgtcca
gagatctgttagaaccatgcgtggctaatcacaagcgatattttctatttgggcatcactacgtatattatgaggattatcgttacgtc
cgtgaaatcgcagtccatgatgtgggaatgattagcacttacgtagatttaaacttaacacttcttaaagatagagagtttatgccgc
tgcaagtatatacaagagacgagctgcgggatacaggattactagactacagtgaaattcaacgccgaaatcaaatgcattcgct
gcgtttttatgacatagacaaggttgtgcaatatgatagcggaacggccattatgcagggcatggctcagtttttccagggacttgg
gaccgcgggccaggccgttggacatgtggttcttggggccacgggagcgctgctttccaccgtacacggatttaccacgttttta
tctaacccatttggggcattggccgtgggattattggttttggcgggactggtagcggccttttttgcgtaccggtacgtgcttaaac
ttaaaacaagcccgatgaaggcattatatccactcacaaccaaggggttaaaacagttaccggaaggaatggatccctttgccga
gaaacccaacgctactgataccccaatagaagaaattggcgactcacaaaacactgaaccgtcggtaaatagcgggtttgatcc
cgataaatttcgagaagcccaggaaatgattaaatttatgacgttagtatctgcggctgagcgccaagaatctaaagcccgcaaa
aaaaataagactagcgcccttttaacttcacgtcttaccggccttgctttacgaaatcgccgaggatactcccgtgttcgcaccgag
aatgtaacgggggtgtaa (SEQ ID NO: 3)
VZV hyperfusogenic gB amino acid sequence:
MSPCGYYSKWRNRDRPEYRRNLRFRRFFSSIHPNAAAGSGFNGPGVFITSVTGV
WLCFLCIFSMFVTAVVSVSPSSFYESLQVEPTQSEDITRSAHLGDGDEIREAIHKSQ
DAETKPTFYVCPPPTGSTIVRLEPPRTCPDYHLGKNFTEGIAVVYKENIAAYKFKA
TVYYKDVIVSTAWAGSSYTQITNRYADRVPIPVSEITDTIDKFGKCSSKATYVRN
NHKVEAFNEDKNPQDMPLIASKYNSVGSKAWHTTNDTYMVAGTPGTYRTGTSV
NCIIEEVEARSIFPYDSFGLSTGDIIYMSPFFGLRDGAYREHSNYAMDRFHQFEGY
RQRDLDTRALLEPAARNFLVTPHLTVGWNWKPKRTEVCSLVKWREVEDVVRDE
YAHNFRFTMKTLSTTFISETNEFNLNQIHLSQCVKEEARAIINRIYTTRYNSSHVRT
GDIQTYLARGGFVVVFQPLLSNSLARLYLQELVRENTNHSPQKHPTRNTRSRRSV
PVELRANRTITTTSSVEFAMLQFTYDHIQEHVNEMLARISSSWCQLQNRERALWS
GLFPINPSALASTILDQRVKARILGDVISVSNCPELGSDTRIILQNSMRVSGSTTRC
YSRPLISIVSLNGSGTVEGQLGTDNELIMSRDLLEPCVANHKRYFLFGHHYVYYE
DYRYVREIAVHDVGMISTYVDLNLTLLKDREFMPLQVYTRDELRDTGLLDYSEI
QRRNQMHSLRFYDIDKVVQYDSGTAIMQGMAQFFQGLGTAGQAVGHVVLGAT
GALLSTVHGFTTFLSNPFGALAVGLLVLAGLVAAFFAYRYVLKLKTSPMKALYP
LTTKGLKQLPEGMDPFAEKPNATDTPIEEIGDSQNTEPSVNSGFDPDKFREAQEMI
KFMTLVSAAERQESKARKKNKTSALLTSRLTGLALRNRRGYSRVRTENVTGV
(SEQ ID NO: 4)
human NIS DNA sequence:
atggaggccgtggagaccggggaacggcccaccttcggagcctgggactacggggtctttgccctcatgctcctggtgtccac
tggcatcgggctgtgggtcgggctggctcggggcgggcagcgcagcgctgaggacttcttcaccgggggccggcgcctggc
ggccctgcccgtgggcctgtcgctgtctgccagcttcatgtcggccgtgcaggtgctgggcgtgccgtcggaggcctatcgcta
tggcctcaagttcctctggatgtgcctgggccagcttctgaactcggtcctcaccgccctgctcttcatgcccgtcttctaccgcct
gggcctcaccagcacctacgagtacctggagatgcgcttcagccgcgcagtgcggctctgcgggactttgcagtacattgtagc
cacgatgctgtacaccggcatcgtaatctacgcaccggccctcatcctgaaccaagtgaccgggctggacatctgggcgtcgct
cctgtccaccggaattatctgcaccttctacacggctgtgggcggcatgaaggctgtggtctggactgatgtgttccaggtcgtgg
tgatgctaagtggcttctgggttgtcctggcacgcggtgtcatgcttgtggggggccccgccaggtgctcacgctggcccaga
accactcccggatcaacctcatggactttaaccctgacccgaggagccgctatacattctggacttttgtggtgggtggcacgttg
gtgtggctctccatgtatggcgtgaaccaggcgcaggtgcagcgctacgtggcttgccgcacagagaagcaggccaagctgg
ccctgctcatcaaccaggtcggcctgttcctgatcgtgtccagcgctgcctgctgtggcatcgtcatgtttgtgttctacactgactg
cgaccctctcctcctgggggcatctctgccccagaccagtacatgcctctgctggtgctggacatcttcgaagatctgcctgga
gtccccgggcttttcctggcctgtgcttacagtggcaccctcagcacagcatccaccagcatcaatgctatggctgcagtcactgt
agaagacctcatcaaacctcggctgcggagcctggcacccaggaaactcgtgattatctccaaggggctctcactcatctacgg
atcggcctgtctcaccgtggcagccctgtcctcactgctcggaggaggtgtccttcagggctccttcaccgtcatgggagtcatc
agcggccccctgctgggagccttcatcttgggaatgttcctgccggcctgcaacacaccgggcgtcctcgcgggactaggcgc
gggcttggcgctgtcgctgtgggtggccttgggcgccacgctgtacccacccagcgagcagaccatgagggtcctgccatcgt
cggctgcccgctgcgtggctctctcagtcaacgcctctggcctcctggacccggctctcctccctgctaacgactccagcaggg
cccccagctcaggaatggacgccagccgacccgccttagctgacagcttctatgccatctcctatctctattacggtgccctggg
cacgctgaccactgtgctgtgcggagccctcatcagctgcctgacaggccccaccaagcgcagcaccctggccccgggattgt
tgtggtgggacctcgcacggcagacagcatcagtggcccccaaggaagaagtggccatcctggatgacaacttggtcaaggg
tcctgaagaactccccactggaaacaagaagccccctggcttcctgcccaccaatgaggatcgtctgtttttcttggggcagaag
gagctggagggggctggctcttggaccccctgtgttggacatgatggtggtcgagaccagcaggagacaaacctctga
(SEQ ID NO: 5)
human NIS amino acid sequence:
MEAVETGERPTFGAWDYGVFALMLLVSTGIGLWVGLARGGQRSAEDFFTGGRR
LAALPVGLSLSASFMSAVQVLGVPSEAYRYGLKFLWMCLGQLLNSVLTALLFMP
VFYRLGLTSTYEYLEMRFSRAVRLCGTLQYIVATMLYTGIVIYAPALILNQVTGL
DIWASLLSTGIICTFYTAVGGMKAVVWTDVFQVVVMLSGFWVVLARGVMLVG
GPRQVLTLAQNHSRINLMDFNPDPRSRYTFWTFVVGGTLVWLSMYGVNQAQVQ
RYVACRTEKQAKLALLINQVGLFLIVSSAACCGIVMFVFYTDCDPLLLGRISAPD
QYMPLLVLDIFEDLPGVPGLFLACAYSGTLSTASTSINAMAAVTVEDLIKPRLRSL
APRKLVIISKGLSLIYGSACLTVAALSSLLGGGVLQGSFTVMGVISGPLLGAFILG
MFLPACNTPGVLAGLGAGLALSLWVALGATLYPPSEQTMRVLPSSAARCVALSV
NASGLLDPALLPANDSSRAPSSGMDASRPALADSFYAISYLYYGALGTLTTVLCG
ALISCLTGPTKRSTLAPGLLWWDLARQTASVAPKEEVAILDDNLVKGPEELPTGN
KKPPGFLPTNEDRLFFLGQKELEGAGSWTPCVGHDGGRDQQETNL (SEQ ID
NO: 6)
human beta-hCG DNA sequence:
atggagatgttccaggggctgctgctgttgctgctgctgagcatgggcgggacatgggcatccaaggagccgcttcggccacg
gtgccgccccatcaatgccaccctggctgtggagaaggagggctgccccgtgtgcatcaccgtcaacaccaccatctgtgccg
gctactgccccaccatgacccgcgtgctgcagggggtcctgccggccctgcctcaggtggtgtgcaactaccgcgatgtgcgc
ttcgagtccatccggctccctggctgcccgcgcggcgtgaaccccgtggtctcctacgccgtggctctcagctgtcaatgtgcac
tctgccgccgcagcaccactgactgcgggggtcccaaggaccaccccttgacctgtgatgacccccgcttccaggactcctctt
cctcaaaggcccctccccccagccttccaagcccatcccgactcccggggccctcggacaccccgatcctcccacaataa
(SEQ ID NO: 7)
human beta-hCG amino acid sequence:
MEMFQGLLLLLLLSMGGTWASKEPLRPRCRPINATLAVEKEGCPVCITVNTTICA
GYCPTMTRVLQGVLPALPQVVCNYRDVRFESIRLPGCPRGVNPVVSYAVALSCQ
CALCRRSTTDCGGPKDHPLTCDDPRFQDSSSSKAPPPSLPSPSRLPGPSDTPILPQ
(SEQ ID NO: 8)
CXCL-13 DNA sequence (accession no. NM_006419):
atgaagttcatctcgacatctctgcttctcatgctgctggtcagcagcctctctccagtccaaggtgttctggaggtctattacacaa
gcttgaggtgtagatgtgtccaagagagctcagtctttatccctagacgcttcattgatcgaattcaaatcttgccccgtgggaatg
gttgtccaagaaaagaaatcatagtctggaagaagaacaagtcaattgtgtgtgtggaccctcaagctgaatggatacaaagaat
gatggaagtattgagaaaaagaagttcttcaactctaccagttccagtgtttaagagaaagattccctaa (SEQ ID NO: 9)
CXCL-13 amino acid sequence:
MKFISTSLLLMLLVSSLSPVQGVLEVYYTSLRCRCVQESSVFIPRRFIDRIQILPRG
NGCPRKEIIVWKKNKSIVCVDPQAEWIQRMMEVLRKRSSSTLPVPVFKRKIP
(SEQ ID NO: 10)
CCL-4 DNA sequence (accession no. NM_002984):
atgaagctctgcgtgactgtcctgtctctcctcatgctagtagctgccttctgctctccagcgctctcagcaccaatgggctcagac
cctcccaccgcctgctgcttttcttacaccgcgaggaagcttcctcgcaactttgtggtagattactatgagaccagcagcctctgc
tcccagccagctgtggtattccaaaccaaaagaagcaagcaagtctgtgctgatcccagtgaatcctgggtccaggagtacgtgt
atgacctggaactgaactaa (SEQ ID NO: 11)
CCL-4 amino acid sequence:
MKLCVTVLSLLMLVAAFCSPALSAPMGSDPPTACCFSYTARKLPRNFVVDYYET
SSLCSQPAVVFQTKRSKQVCADPSESWVQEYVYDLELN (SEQ ID NO: 12)
PEDF DNA sequence (accession no. NM_002615):
atgcaggccctggtgctactcctctgcattggagccctcctcgggcacagcagctgccagaaccctgccagccccccggagga
gggctccccagaccccgacagcacaggggcgctggtggaggaggaggatcctttcttcaaagtccccgtgaacaagctggca
gcggctgtctccaacttcggctatgacctgtaccgggtgcgatccagcatgagccccacgaccaacgtgctcctgtctcctctca
gtgtggccacggccctctcggccctctcgctgggagcggagcagcgaacagaatccatcattcaccgggctctctactatgact
tgatcagcagcccagacatccatggtacctataaggagctccttgacacggtcactgcccgccagaagaacctcaagagtgcct
cccggatcgtctttgagaagaagctgcgcataaaatccagctttgtggcacctctggaaaagtcatatgggaccaggcccagag
tcctgacgggcaaccctcgcttggacctgcaagagatcaacaactgggtgcaggcgcagatgaaagggaagctcgccaggtc
cacaaaggaaattcccgatgagatcagcattctccttctcggtgtggcgcacttcaaggggcagtgggtaacaaagtttgactcc
agaaagacttccctcgaggatttctacttggatgaagagaggaccgtgagggtccccatgatgtcggaccctaaggctgttttac
gctatggcttggattcagatctcagctgcaagattgcccagctgcccttgaccggaagcatgagtatcatcttcttcctgcccctga
aagtgacccagaatttgaccttgatagaggagagcctcacctccgagttcattcatgacatagaccgagaactgaagaccgtgc
aggcggtcctcactgtccccaagctgaagctgagttacgaaggcgaagtcaccaagtccctgcaggagatgaagctgcaatcc
ttgtttgattcaccagactttagcaagatcacaggcaaacccatcaagctgactcaggtggaacaccgggctggctttgagtgga
acgaggatggggcgggaaccacccccagcccagggctgcagcctgcccacctcaccttcccgctggactatcaccttaacca
gcctttcatcttcgtactgagggacacagacacaggggcccttctcttcattggcaagattctggaccccaggggcccctaa
(SEQ ID NO: 13)
PEDF amino acid sequence:
MQALVLLLCIGALLGHSSCQNPASPPEEGSPDPDSTGALVEEEDPFFKVPVNKLA
AAVSNFGYDLYRVRSSMSPTTNVLLSPLSVATALSALSLGAEQRTESIIHRALYY
DLISSPDIHGTYKELLDTVTARQKNLKSASRIVFEKKLRIKSSFVAPLEKSYGTRPR
VLTGNPRLDLQEINNWVQAQMKGKLARSTKEIPDEISILLLGVAHFKGQWVTKF
DSRKTSLEDFYLDEERTVRVPMMSDPKAVLRYGLDSDLSCKIAQLPLTGSMSIIFF
LPLKVTQNLTLIEESLTSEFIHDIDRELKTVQAVLTVPKLKLSYEGEVTKSLQEMK
LQSLFDSPDFSKITGKPIKLTQVEHRAGFEWNEDGAGTTPSPGLQPAHLTFPLDYH
LNQPFIFVLRDTDTGALLFIGKILDPRGP (SEQ ID NO: 14)
single chain IL-12 DNA sequence:
atgtgtcaccagcagttggtcatctcttggttttccctggtttttctggcatctcccctcgtggccatatgggaactgaagaaagatgt
ttatgtcgtagaattggattggtatccggatgcccctggagaaatggtggtcctcacctgtgacacccctgaagaagatggtatca
cctggaccttggaccagagcagtgaggtcttaggctctggcaaaaccctgaccatccaagtcaaagagtttggagatgctggcc
agtacacctgtcacaaaggaggcgaggttctaagccattcgctcctgctgcttcacaaaaaggaagatggaatttggtccactga
tattttaaaggaccagaaagaacccaaaaataagacctttctaagatgcgaggccaagaattattctggacgtttcacctgctggtg
gctgacgacaatcagtactgatttgacattcagtgtcaaaagcagcagaggctcttctgacccccaaggggtgacgtgcggagc
tgctacactctctgcagagagagtcagaggggacaacaaggagtatgagtactcagtggagtgccaggaggacagtgcctgc
ccagctgctgaggagagtctgcccattgaggtcatggtggatgccgttcacaagctcaagtatgaaaactacaccagcagcttct
tcatcagggacatcatcaaacctgacccacccaagaacttgcagctgaagccattaaagaattctcggcaggtggaggtcagct
gggagtaccctgacacctggagtactccacattcctacttctccctgacattctgogttcaggtccagggcaagagcaagagaga
aaagaaagatagagtcttcacggacaagacctcagccacggtcatctgccgcaaaaatgccagcattagcgtgcgggcccag
gaccgctactatagctcatcttggagcgaatgggcatctgtgccctgcagtgttcctggagtaggggtacctggggtgggcgcc
agaaacctccccgtggccactccagacccaggaatgttcccatgccttcaccactcccaaaacctgctgagggccgtcagcaa
catgctccagaaggccagacaaactctagaattttacccttgcacttctgaagagattgatcatgaagatatcacaaaagataaaa
ccagcacagtggaggcctgtttaccattggaattaaccaagaatgagagttgcctaaattccagagagacctctttcataactaatg
ggagttgcctggcctccagaaagacctcttttatgatggccctgtgccttagtagtatttatgaagacttgaagatgtaccaggtgg
agttcaagaccatgaatgcaaagctgctgatggatcctaagaggcagatctttctagatcaaaacatgctggcagttattgatgag
ctgatgcaggccctgaatttcaacagtgagactgtgccacaaaaatcctcccttgaagaaccggatttttataaaactaaaatcaag
ctctgcatacttcttcatgctttcagaattcgggcagtgactattgatagagtgatgagctatctgaatgcttcctaa (SEQ ID
NO: 15)
single chain IL-12 amino acid sequence:
MCHQQLVISWFSLVFLASPLVAIWELKKDVYVVELDWYPDAPGEMVVLTCDTP
EEDGITWTLDQSSEVLGSGKTLTIQVKEFGDAGQYTCHKGGEVLSHSLLLLHKKE
DGIWSTDILKDQKEPKNKTFLRCEAKNYSGRFTCWWLTTISTDLTFSVKSSRGSS
DPQGVTCGAATLSAERVRGDNKEYEYSVECQEDSACPAAEESLPIEVMVDAVH
KLKYENYTSSFFIRDIIKPDPPKNLQLKPLKNSRQVEVSWEYPDTWSTPHSYFSLT
FCVQVQGKSKREKKDRVFTDKTSATVICRKNASISVRAQDRYYSSSWSEWASVP
CSVPGVGVPGVGARNLPVATPDPGMFPCLHHSQNLLRAVSNMLQKARQTLEFY
PCTSEEIDHEDITKDKTSTVEACLPLELTKNESCLNSRETSFITNGSCLASRKTSFM
MALCLSSIYEDLKMYQVEFKTMNAKLLMDPKRQIFLDQNMLAVIDELMQALNF
NSETVPQKSSLEEPDFYKTKIKLCILLHAFRIRAVTIDRVMSYLNAS (SEQ ID
NO: 16)
IL15RAsushi/IL 15 DNA sequence:
atggccccgcggcgggcgcgcggctgccggaccctcggtctcccggcgctgctactgctgctgctgctccggccgccggcg
acgcggggcatcacgtgccctccccccatgtccgtggaacacgcagacatctgggtcaagagctacagcttgtactccaggga
gcggtacatttgtaactctggtttcaagcgtaaagccggcacgtccagcctgacggagtgcgtgttgaacaaggccacgaatgtc
gcccactggacaacccccagtctcaaatgcattagaagcggcggcagcggcggcggcggcagcggcggcggcagcggcg
gcggcggcagcctgcagaactgggtgaatgtaataagtgatttgaaaaaaattgaagatcttattcaatctatgcatattgatgcta
ctttatatacggaaagtgatgttcaccccagttgcaaagtaacagcaatgaagtgctttctcttggagttacaagttatttcacttgagt
ccggagatgcaagtattcatgatacagtagaaaatctgatcatcctagcaaacaacagtttgtcttctaatgggaatgtaacagaat
ctggatgcaaagaatgtgaggaactggaggaaaaaaatattaaagaatttttgcagagttttgtacatattgtccaaatgttcatcaa
cacttcttga (SEQ ID NO: 17)
IL15RAsushi/IL 15 amino acid sequence:
MAPRRARGCRTLGLPALLLLLLLRPPATRGITCPPPMSVEHADIWVKSYSLYSRE
RYICNSGFKRKAGTSSLTECVLNKATNVAHWTTPSLKCIRSGGSGGGGSGGGSG
GGGSLQNWVNVISDLKKIEDLIQSMHIDATLYTESDVHPSCKVTAMKCFLLELQV
ISLESGDASIHDTVENLIILANNSLSSNGNVTESGCKECEELEEKNIKEFLQSFVHIV
QMFINTS (SEQ ID NO: 18)
cleavage resistant BDNF DNA sequence:
atgaccatccttttccttactatggttatttcatactttggttgcatgaaggctgcccccatgaaagaagcaaacatccgaggacaag
gtggcttggcctacccaggtgtgcggacccatgggactctggagagcgtgaatgggcccaaggcaggttcaagaggcttgac
atcattggctgacactttcgaacacgtgatagaagagctgttggatgaggaccagaaagttcggcccaatgaagaaaacaataa
ggacgcagacttgtacacgtccagggtgatgctcagtagtcaagtgcctttggagcctcctcttctctttctgctggaggaatacaa
aaattacctagatgctgcaaacatgtccatggcggtcgcggcccactctgaccctgcccgccgaggggagctcagcgtgtgtg
acagtattagtgagtgggtaacggcggcagacaaaaagactgcagtggacatgtcggggggacggtcacagtccttgaaaa
ggtccctgtatcaaaaggccaactgaagcaatacttctacgagaccaagtgcaatcccatgggttacacaaaagaaggctgcag
gggcatagacaaaaggcattggaactcccagtgccgaactacccagtcgtacgtgcgggcccttaccatggatagcaaaaaga
gaattggctggcgattcataaggatagacacttcttgtgtatgtacattgaccattaaaaggggaagataa (SEQ ID
NO: 19)
cleavage resistant BDNF amino acid sequence:
MTILFLTMVISYFGCMKAAPMKEANIRGQGGLAYPGVRTHGTLESVNGPKAGSR
GLTSLADTFEHVIEELLDEDQKVRPNEENNKDADLYTSRVMLSSQVPLEPPLLFL
LEEYKNYLDAANMSMAVAAHSDPARRGELSVCDSISEWVTAADKKTAVDMSG
GTVTVLEKVPVSKGQLKQYFYETKCNPMGYTKEGCRGIDKRHWNSQCRTTQSY
VRALTMDSKKRIGWRFIRIDTSCVCTLTIKRGR (SEQ ID NO: 20)
Functional UL131 DNA sequence:
atgcggctgtgtcgggtgtggctgtctgtttgtctgtgcgccgtggtgctgggtcagtgccagcgggaaaccgcggaaaaaaac
gattattaccgagtaccgcattactgggacgcgtgctctcgcgcgctgcccgaccaaacccgttacaagtatgtggaacagctcg
tggacctcacgttgaactaccactacgatgcgagccacggcttggacaactttgacgtgctcaagaggtgagggtacgcgctaa
agatgcatgacaacgggaaggtaagggcgaacgggtaacgggtaagtaaccgcatggggtatgaaatgacgttcggaacctg
tgcttgcagaatcaacgtgaccgaggtgtcgttgctcatcagcgactttagacgtcagaaccgtcgcggcggcaccaacaaaag
gaccacgttcaacgccgccggttcgctggcgccacacgcccggagcctcgagttcagcgtgcggctctttgccaactag
(SEQ ID NO: 21)
Functional UL131 amino acid sequence:
MRLCRVWLSVCLCAVVLGQCQRETAEKNDYYRVPHYWDACSRALPDQTRYKY
VEQLVDLTLNYHYDASHGLDNFDVLKR*GYALKMHDNGKVRANG*RVS
NRMGYEMTFGTCACRINVTEVSLLISDFRRQNRRGGTNKRTTFNAAGSLAPHAR
SLEFSVRLFAN (SEQ ID NO: 22)
CD::UPRT DNA sequence:
atggtcacaggaggcatggcttcaaagtgggaccagaagggcatggacattgcctatgaggaggctgctctgggctacaagg
agggaggggtcccaattggtggctgcctcatcaacaacaaggatggcagtgtcctgggcaggggccacaacatgaggttcca
gaagggcagtgccaccctgcatggggagatcagcaccctggagaactgtggcaggctggagggcaaggtctacaaggacac
cactctgtacaccaccctcagcccttgtgacatgtgcacaggggccatcatcatgtatggcattcccaggtgtgtggtgggagag
aatgtcaacttcaagtcaaaaggagagaagtacctccagaccaggggccatgaggtggttgtggtggatgatgagaggtgcaa
gaagattatgaagcagttcattgatgagagaccccaggactggtttgaggacattggggaggcctctgagcccttcaagaatgtg
tacctcctcccccagaccaaccaactcctgggactctacaccatcatcaggaacaagaacaccaccaggccagacttcatcttct
acagtgacaggatcatcaggctcctggtggaggagggcctcaaccacctccctgtgcagaagcagattgtggagactgacacc
aatgagaactttgagggagtgtctttcatgggcaagatttgtggggtgtccattgtgagggctggggagagcatggagcagggc
ctgagggactgttgcaggagtgtgaggattggcaagatcctgatccagagggatgaggagactgccctgcccaagctgttctat
gagaagctccctgaagacatctctgagaggtatgtcttcctcctggaccccatgctggcaactggaggctctgcaatcatggcca
ctgaggtgctcatcaagaggggagtcaagcctgagaggatctacttcctcaacctcatctgctcaaaggagggcattgagaagt
accatgctgccttccctgaagtgaggattgtcactggggctctggacaggggcctggatgagaacaagtacctggtccctggcc
tgggagactttggggacagatactactgtgtctaa (SEQ ID NO: 23)
CD::UPRT amino acid sequence:
MVTGGMASKWDQKGMDIAYEEAALGYKEGGVPIGGCLINNKDGSVLGRGHNM
RFQKGSATLHGEISTLENCGRLEGKVYKDTTLYTTLSPCDMCTGAIIMYGIPRCV
VGENVNFKSKGEKYLQTRGHEVVVVDDERCKKIMKQFIDERPQDWFEDIGEASE
PFKNVYLLPQTNQLLGLYTIIRNKNTTRPDFIFYSDRIIRLLVEEGLNHLPVQKQIV
ETDTNENFEGVSFMGKICGVSIVRAGESMEQGLRDCCRSVRIGKILIQRDEETALP
KLFYEKLPEDISERYVFLLDPMLATGGSAIMATEVLIKRGVKPERIYFLNLICSKE
GIEKYHAAFPEVRIVTGALDRGLDENKYLVPGLGDFGDRYYCV (SEQ ID NO: 24)
Paramyxovirus H polypeptide DNA sequence:
atggactataaagacgatgatgacaaatcaccacaacgagaccggataaatgccttctacaaagataacccccatcccaaggga
agtaggatagtcattaacagagaacatcttatgattgatagaccttatgttttgctggctgttctgtttgtcatgtttctgagcttgatcg
ggttgctagccattgcaggaattcgacttcatcgggcagccatctacaccgcagagatccataaaagcctcagcaccaatctaga
tgtaactaactcaatcgagcatcaggtcaaggacgtgctgacaccactcttcaaaatcatcggtgatgaagtgggcctgaggaca
cctcagagattcactgacctagtgaaattcatctctgacaagattaaattccttaatccggatagggagtacgacttcagagatctca
cttggtgtatcaacccgccagagagaatcaaattggattatgatcaatactgtgcagatgtggctgctgaagagctcatgaatgca
ttggtgaactcaactctactggagaccagaacaaccaatcagttcctagctgtctcaaagggaaactgctcagggcccactacaa
tcagaggtcaattctcaaacatgtcgctgtccctgttagacttgtatttaggtcgaggttacaatgtgtcatctatagtcactatgacat
cccagggaatgtatgggggaacttacctagtggaaaagcctaatctgagcagcaaaaggtcagagttgtcacaactgagcatgt
accgagtgtttgaagtaggtgttatcagaaatccgggtttgggggctccggtgttccatatgacaaactatcttgagcaaccagtca
gtaatgatctcagcaactgtatggtggctttgggggagctcaaactcgcagccctttgtcacggggaagattctatcacaattccct
atcagggatcagggaaaggtgtcagcttccagctcgtcaagctaggtgtctggaaatccccaaccgacatgcaatcctgggtcc
ccttatcaacggatgatccagtgatagacaggctttacctctcatctcacagaggtgttatcgctgacaaccaagcaaaatgggct
gtcccgacaacacgaacagatgacaagttgcgaatggagacatgcttccaacaggcgtgtaagggtaaaatccaagcactctg
cgagaatcccgagtgggcaccattgaaggataacaggattccttcatacggggtcttgtctgttgatctgagtctgacagttgagc
ttaaaatcaaaattgcttcgggattcgggccattgatcacacacggttcagggatggacctatacaaatccaaccacaacaatgtg
tattggctgactatcccgccaatgaagaacctagccttaggtgtaatcaacacattggagtggataccgagattcaaggttagtcc
ctacctcttcactgtcccaattaaggaagcaggcggagactgccatgccccaacatacctacctgcggaggtggatggtgatgtc
aaactcagttccaatctggtgattctacctggtcaagatctccaatatgttttggcaacctacgatacttccagggttgaacatgctgt
ggtttattacgtttacagcccaagccgctcattttcttacttttatccttttaggttgcctataaagggggtccccatcgaattacaagtg
gaatgcttcacatgggaccaaaaactctggtgccgtcacttctgtgtgcttgcggactcagaatctggtggacatatcactcactct
gggatggtgggcatgggagtcagctgcacagtcacccgggaagatggaaccaatcgcagatag (SEQ ID NO: 25)
Paramyxovirus H polypeptide amino acid sequence:
MDYKDDDDKSPQRDRINAFYKDNPHPKGSRIVINREHLMIDRPYVLLAVLFVMF
LSLIGLLAIAGIRLHRAAIYTAEIHKSLSTNLDVTNSIEHQVKDVLTPLFKIIGDEVG
LRTPQRFTDLVKFISDKIKFLNPDREYDFRDLTWCINPPERIKLDYDQYCADVAA
EELMNALVNSTLLETRTTNQFLAVSKGNCSGPTTIRGQFSNMSLSLLDLYLGRGY
NVSSIVTMTSQGMYGGTYLVEKPNLSSKRSELSQLSMYRVFEVGVIRNPGLGAP
VFHMTNYLEQPVSNDLSNCMVALGELKLAALCHGEDSITIPYQGSGKGVSFQLV
KLGVWKSPTDMQSWVPLSTDDPVIDRLYLSSHRGVIADNQAKWAVPTTRTDDK
LRMETCFQQACKGKIQALCENPEWAPLKDNRIPSYGVLSVDLSLTVELKIKIASG
FGPLITHGSGMDLYKSNHNNVYWLTIPPMKNLALGVINTLEWIPRFKVSPYLFTV
PIKEAGGDCHAPTYLPAEVDGDVKLSSNLVILPGQDLQYVLATYDTSRVEHAVV
YYVYSPSRSFSYFYPFRLPIKGVPIELQVECFTWDQKLWCRHFCVLADSESGGHIT
HSGMVGMGVSCTVTREDGTNRR (SEQ ID NO: 26)
Paramyxovirus F polypeptide DNA sequence:
atgggtctcaaggtgaacgtctctgccatattcatggcagtactgttaactctccaaacacccaccggtcaaatccattggggcaat
ctctctaagataggggggtaggaataggaagtgcaagctacaaagttatgactcgttccagccatcaatcattagtcataaaatta
atgcccaatataactctcctcaataactgcacgagggtagagattgcagaatacaggagactactgagaacagttttggaaccaa
ttagagatgcacttaatgcagtgacccagaatataagaccggttcagagtgtagcttcaagtaggagacacaagagatttgcggg
agtagtcctggcaggtgcggccctaggcgttgccacagctgctcagataacagccggcattgcacttcaccagtccatgctgaa
ctctcaagccatcgacaatctgagagcgagcctggaaactactaatcaggcaattgaggcaatcagacaagcagggcaggag
atgatattggctgttcagggtgtccaagactacatcaataatgagctgataccgtctatgaaccaactatcttgtgatttaatcggcc
agaagctcgggctcaaattgctcagatactatacagaaatcctgtcattatttggccccagcttacgggaccccatatctgcggag
atatctatccaggctttgagctatgcgcttggaggagacatcaataaggtgttagaaaagctcggatacagtggaggtgatttact
gggcatcttagagagcagaggaataaaggcccggataactcacgtcgacacagagtcctacttcattgtcctcagtatagcctat
ccgacgctgtccgagattaagggggtgattgtccaccggctagagggggtctcgtacaacataggctctcaagagtggtatacc
actgtgcccaagtatgttgcaacccaagggtaccttatctcgaattttgatgagtcatcgtgtactttcatgccagaggggactgtgt
gcagccaaaatgccttgtacccgatgagtcctctgctccaagaatgcctccgggggtccaccaagtcctgtgctcgtacactcgt
atccgggtcttttgggaaccggttcattttatcacaagggaacctaatagccaattgtgcatcaatcctttgcaagtgttacacaaca
ggaacgatcattaatcaagaccctgacaagatcctaacatacattgctgccgatcactgcccggtagtcgaggtgaacggcgtg
accatccaagtcgggagcaggaggtatccagacgctgtgtacttgcacagaattgacctcggtcctcccatatcattggagaggt
tggacgtagggacaaatctggggaatgcaattgctaagttggaggatgccaaggaattgttggagtcatcggaccagatattga
ggagtatgaaaggtttatcgagcactagcatagtctacatcctgattgcagtgtgtcttggagggttgatagggatccccgctttaat
atgttgctgcagggggcgttgtaacaaaaagggagaacaagttggtatgtcaagaccaggcctaaagcctgatcttacgggaac
atcaaaatcctatgtaaggtcgctctga (SEQ ID NO: 27)
Paramyxovirus F polypeptide amino sequence:
MGLKVNVSAIFMAVLLTLQTPTGQIHWGNLSKIGVVGIGSASYKVMTRSSHQSL
VIKLMPNITLLNNCTRVEIAEYRRLLRTVLEPIRDALNAVTQNIRPVQSVASSRRH
KRFAGVVLAGAALGVATAAQITAGIALHQSMLNSQAIDNLRASLETTNQAIEAIR
QAGQEMILAVQGVQDYINNELIPSMNQLSCDLIGQKLGLKLLRYYTEILSLFGPSL
RDPISAEISIQALSYALGGDINKVLEKLGYSGGDLLGILESRGIKARITHVDTESYFI
VLSIAYPTLSEIKGVIVHRLEGVSYNIGSQEWYTTVPKYVATQGYLISNFDESSCT
FMPEGTVCSQNALYPMSPLLQECLRGSTKSCARTLVSGSFGNRFILSQGNLIANC
ASILCKCYTTGTIINQDPDKILTYIAADHCPVVEVNGVTIQVGSRRYPDAVYLHRI
DLGPPISLERLDVGTNLGNAIAKLEDAKELLESSDQILRSMKGLSSTSIVYILIAVC
LGGLIGIPALICCCRGRCNKKGEQVGMSRPGLKPDLTGTSKSYVRSL (SEQ ID
NO: 28)
atggactataaagacgatgatgacaaatcaccacaacgagaccggataaatgccttctacaaagataacccccatcccaaggga
agtaggatagtcattaacagagaacatcttatgattgatagaccttatgttttgctggctgttctgtttgtcatgtttctgagcttgatcg
ggttgctagccattgcaggaattcgacttcatcgggcagccatctacaccgcagagatccataaaagcctcagcaccaatctaga
tgtaactaactcaatcgagcatcaggtcaaggacgtgctgacaccactcttcaaaatcatcggtgatgaagtgggcctgaggaca
cctcagagattcactgacctagtgaaattcatctctgacaagattaaattccttaatccggatagggagtacgacttcagagatctca
cttggtgtatcaacccgccagagagaatcaaattggattatgatcaatactgtgcagatgtggctgctgaagagctcatgaatgca
ttggtgaactcaactctactggagaccagaacaaccaatcagttcctagctgtctcaaagggaaactgctcagggcccactacaa
tcagaggtcaattctcaaacatgtcgctgtccctgttagacttgtatttaggtcgaggttacaatgtgtcatctatagtcactatgacat
cccagggaatgtatgggggaacttacctagtggaaaagcctaatctgagcagcaaaaggtcagagttgtcacaactgagcatgt
accgagtgtttgaagtaggtgttatcagaaatccgggtttgggggctccggtgttccatatgacaaactatcttgagcaaccagtca
gtaatgatctcagcaactgtatggtggctttgggggagctcaaactcgcagccctttgtcacggggaagattctatcacaattccct
atcagggatcagggaaaggtgtcagcttccagctcgtcaagctaggtgtctggaaatccccaaccgacatgcaatcctgggtcc
ccttatcaacggatgatccagtgatagacaggctttacctctcatctcacagaggtgttatcgctgacaaccaagcaaaatgggct
gtcccgacaacacgaacagatgacaagttgcgaatggagacatgcttccaacaggcgtgtaagggtaaaatccaagcactctg
cgagaatcccgagtgggcaccattgaaggataacaggattccttcatacggggtcttgtctgttgatctgagtctgacagttgagc
ttaaaatcaaaattgcttcgggattcgggccattgatcacacacggttcagggatggacctatacaaatccaaccacaacaatgtg
tattggctgactatcccgccaatgaagaacctagccttaggtgtaatcaacacattggagtggataccgagattcaaggttagtcc
cgcgctcttcaatgtcccaattaaggaagcaggcggagactgccatgccccaacatacctacctgcggaggtggatggtgatgt
caaactcagttccaatctggtgattctacctggtcaagatctccaatatgttttggcaacctacgatacttccgcggttgaacatgctg
tggtttattacgtttacagcccaagccgcttaagttcttacttttatccttttaggttgcctataaagggggtccccatcgaattacaagt
ggaatgcttcacatgggaccaaaaactctggtgccgtcacttctgtgtgcttgcggactcagaatctggtggacatatcactcactc
tgggatggtgggcatgggagtcagctgcacagtcacccgggaagatggaaccaatatcgagggaagggcggcccagccgg
ccatggccgaggtgcaactgcagcagtctggggctgaactggtgaagcctggggcttcagtgaagttgtcctgcaaggcttccg
gctacaccttcaccagccactggatgcactgggtgaagcagagggctggacaaggccttgagtggatcggagagtttaatccc
agcaacggccgtactaactacaatgagaaattcaagagcaaggccacactgactgtagacaaatcctccagcacagcctacat
gcaactcagcagcctgacatctgaggactctgcggtctattactgtgccagtcgggactatgattacgacggacggtactttgact
actggggccaagggaccacggtcaccgtctcctcaggtggcggtggctcgggcggtggtgggtcgggtggtggcggatctg
acatcgagctcacccagtctccagcaatcatgtctgcatctccaggggagaaggtcactatgacctgcagtgccagctcaagtgt
aacttacatgtattggtaccagcagaagccaggatcctcccccagactcctgatttatgacacatccaacctggcttctggagtcc
ctgttcgtttcagtggcagtgggtctgggacctcttactctctcacaatcagccgaatggaggctgaagatgctgccacttattact
gccagcagtggagtagtcacatattcacgttcggctcggggacagaactcgagatcaaacgggcggccgcaagaggatcgca
tcaccatcaccatcactga (SEQ ID NO: 29)
EGFR retargeted Paramyxovirus H polypeptide amino acid sequence:
MDYKDDDDKSPQRDRINAFYKDNPHPKGSRIVINREHLMIDRPYVLLAVLFVMF
LSLIGLLAIAGIRLHRAAIYTAEIHKSLSTNLDVTNSIEHQVKDVLTPLFKIIGDEVG
LRTPQRFTDLVKFISDKIKFLNPDREYDFRDLTWCINPPERIKLDYDQYCADVAA
EELMNALVNSTLLETRTTNQFLAVSKGNCSGPTTIRGQFSNMSLSLLDLYLGRGY
NVSSIVTMTSQGMYGGTYLVEKPNLSSKRSELSQLSMYRVFEVGVIRNPGLGAP
VFHMTNYLEQPVSNDLSNCMVALGELKLAALCHGEDSITIPYQGSGKGVSFQLV
KLGVWKSPTDMQSWVPLSTDDPVIDRLYLSSHRGVIADNQAKWAVPTTRTDDK
LRMETCFQQACKGKIQALCENPEWAPLKDNRIPSYGVLSVDLSLTVELKIKIASG
FGPLITHGSGMDLYKSNHNNVYWLTIPPMKNLALGVINTLEWIPRFKVSPALFNV
PIKEAGGDCHAPTYLPAEVDGDVKLSSNLVILPGQDLQYVLATYDTSAVEHAVV
YYVYSPSRLSSYFYPFRLPIKGVPIELQVECFTWDQKLWCRHFCVLADSESGGHIT
HSGMVGMGVSCTVTREDGTNIEGRAAQPAMAEVQLQQSGAELVKPGASVKLSC
KASGYTFTSHWMHWVKQRAGQGLEWIGEFNPSNGRTNYNEKFKSKATLTVDKS
SSTAYMQLSSLTSEDSAVYYCASRDYDYDGRYFDYWGQGTTVTVSSGGGGSGG
GGSGGGGSDIELTQSPAIMSASPGEKVTMTCSASSSVTYMYWYQQKPGSSPRLLI
YDTSNLASGVPVRFSGSGSGTSYSLTISRMEAEDAATYYCQQWSSHIFTFGSGTE
LEIKRAAARGSHHHHHH (SEQ ID NO: 30)

This document also provides methods and materials for using combinations of recombinant herpesviruses described herein (e.g., a recombinant HSV having oncolytic anti-cancer activity and a recombinant CMV having oncolytic anti-cancer activity, or a recombinant VZV having oncolytic anti-cancer activity and a recombinant CMV having oncolytic anti-cancer activity). In some cases, a recombinant herpesvirus provided herein can be used for treating a mammal having, or at risk of having, cancer. For example, methods for treating a mammal having, or at risk of having, cancer can include administering a first recombinant herpesviruses described herein and a second recombinant herpesvirus described herein to the mammal, where the first and second recombinant herpesviruses are from different herpesvirus subfamilies. In some cases, first and second recombinant herpesviruses described herein can be administered to a mammal to reduce the number of cancer cells in the mammal (e.g., suppress and/or delay tumor growth) and/or to increase survival of the mammal, where the first and second recombinant herpesviruses are from different herpesvirus subfamilies. For example, first and second recombinant herpesviruses described herein can be administered to a mammal to induce syncytia formation of cancer cells within a mammal, where the first and second recombinant herpesviruses are from different herpesvirus subfamilies. In some cases, first and second recombinant herpesviruses described herein can be administered to a mammal to induce cell death in a cell of the mammal (e.g., in an infected cell of the mammal), where the first and second recombinant herpesviruses are from different herpesvirus subfamilies.

A first recombinant herpesvirus provided herein (e.g., a recombinant HSV having oncolytic anti-cancer activity or a recombinant VZV having oncolytic anti-cancer activity) and a second recombinant herpesvirus provided herein (e.g., a recombinant CMV having oncolytic anti-cancer activity) can be administered to a mammal separately or together. For example, in some cases, a recombinant alpha herpesvirus provided herein (e.g., a recombinant HSV having oncolytic anti-cancer activity or a recombinant VZV having oncolytic anti-cancer activity) can be administered to a mammal, and subsequently a recombinant beta herpesvirus provided herein (e.g., a recombinant CMV having oncolytic anti-cancer activity) can be administered to the mammal. For example, in some cases, a recombinant beta herpesvirus provided herein (e.g., a recombinant CMV having oncolytic anti-cancer activity) can be administered to a mammal, and subsequently a recombinant alpha herpesvirus provided herein (e.g., a recombinant HSV having oncolytic anti-cancer activity or a recombinant VZV having oncolytic anti-cancer activity) can be administered to the mammal. In some cases, a recombinant alpha herpesvirus provided herein (e.g., a recombinant HSV having oncolytic anti-cancer activity or a recombinant VZV having oncolytic anti-cancer activity) and a recombinant beta herpesvirus provided herein (e.g., a recombinant CMV having oncolytic anti-cancer activity) can be administered simultaneously to the mammal.

Any appropriate mammal having, or at risk of having, cancer can be treated as described herein. For example, humans, non-human primates, monkeys, horses, bovine species, porcine species, dogs, cats, rabbits, mice, and rats having cancer can be treated for cancer as described herein. In some cases, a human having cancer can be treated. In some cases, a mammal (e.g., a human) treated as described herein is not a natural host of a herpesvirus used to generate a recombinant herpesvirus described herein (e.g., a recombinant herpesvirus having oncolytic anti-cancer activity).

A mammal having any appropriate type of cancer can be treated as described herein (e.g., treated with first and second recombinant herpesviruses described herein, such as a recombinant HSV having oncolytic anti-cancer activity and a recombinant CMV having oncolytic anti-cancer activity, or a recombinant VZV having oncolytic anti-cancer activity and a recombinant CMV having oncolytic anti-cancer activity). In some cases, a cancer treated as described herein can include one or more solid tumors. Examples of cancers that can be treated as described herein include, without limitation, brain cancers (e.g., glioblastoma), pancreatic cancers (e.g., pancreatic adenocarcinoma), bile duct cancers (e.g., cholangiocarcinoma), lung cancers (e.g., mesothelioma), skin cancers (e.g., melanoma), prostate cancers, breast cancers, ovarian cancers, liver cancers, colorectal cancers, stomach cancers, kidney cancers, and cancers of the head and neck. For example, a cancer treated as described herein can be a glioblastoma. For example, a cancer treated as described herein can be a pancreatic adenocarcinoma. For example, a cancer treated as described herein can be an ovarian cancer.

In some cases, methods described herein also can include identifying a mammal as having cancer. Examples of methods for identifying a mammal as having cancer include, without limitation, physical examination, laboratory tests (e.g., blood and/or urine), biopsy, imaging tests (e.g., X-ray, PET/CT, MRI, and/or ultrasound), nuclear medicine scans (e.g., bone scans), endoscopy, and/or genetic tests. Once identified as having cancer, a mammal can be administered a first recombinant herpesvirus and a second recombinant herpesvirus described herein (e.g., a recombinant HSV having oncolytic anti-cancer activity and a recombinant CMV having oncolytic anti-cancer activity, or a recombinant VZV having oncolytic anti-cancer activity and a recombinant CMV having oncolytic anti-cancer activity).

First and second recombinant herpesviruses described herein (e.g., a recombinant HSV having oncolytic anti-cancer activity and a recombinant CMV having oncolytic anti-cancer activity, or a recombinant VZV having oncolytic anti-cancer activity and a recombinant CMV having oncolytic anti-cancer activity) can be administered by any appropriate route (e.g., intratumorally, intravenously, intramuscularly, subcutaneously, orally, intranasally, by inhalation, transdermally, or parenterally) to a mammal. In some cases, a first recombinant herpesvirus described herein and a second recombinant herpesvirus described herein can be administered to a mammal (e.g., a human) by direct injection into a group of cancer cells (e.g., a tumor) or intravenous delivery to cancer cells. In some cases, first and second herpesviruses provided herein can be directly administered into a tumor (e.g., a breast cancer tumor) that is palpable through the skin. Ultrasound guidance also can be used in such a method. Alternatively, direct administration of a virus can be achieved via a catheter line or other medical access device, and can be used in conjunction with an imaging system to localize a group of cancer cells. In such methods, an implantable dosing device can be placed in proximity to a group of cancer cells using a guidewire inserted into the medical access device. In some cases, effective doses of first and second recombinant herpesviruses provided herein (e.g., a recombinant HSV having oncolytic anti-cancer activity and a recombinant CMV having oncolytic anticancer activity, or a recombinant VZV having oncolytic anti-cancer activity and a recombinant CMV having oncolytic anti-cancer activity) can be directly administered to a group of cancer cells that is visible in an exposed surgical field.

While dosages administered will vary from patient to patient (e.g., depending upon the size of a tumor), an effective dose can be determined by setting as a lower limit the concentration of virus proven to be safe and escalating to higher doses of up to 10¹² pfu, while monitoring for a reduction in cancer cell growth along with the presence of any deleterious side effects. A therapeutically effective dose typically provides at least a 10% reduction in the number of cancer cells or in tumor size. Escalating dose studies can be used to obtain a desired effect for a given viral treatment (see, e.g., Nies and Spielberg, “Principles of Therapeutics,” In Goodman & Gilman's The Pharmacological Basis of Therapeutics, eds. Hardman, et al., McGraw-Hill, NY, 1996, pp 43-62).

Recombinant herpesviruses provided herein can be delivered in a dose ranging from, for example, about 10³ transducing units per kg (TU/kg) to about 10¹² TU/kg (e.g., about 10⁵ TU/kg to about 10¹² TU/kg, about 10⁶ TU/kg to about 10¹¹ TU/kg, or about 10⁶ TU/kg to about 10¹⁰ TU/kg). In some cases, a therapeutically effective dose can be provided in repeated doses. Repeat dosing can be appropriate in cases in which observations of clinical symptoms or tumor size or monitoring assays indicate either that a group of cancer cells or tumor has stopped shrinking or that the degree of viral activity is declining while the tumor is still present. Repeat doses can be administered by the same route as initially used or by another route. A therapeutically effective dose can be delivered in several discrete doses (e.g., days or weeks apart) and in one embodiment, one to about twelve doses are provided. Alternatively, a therapeutically effective dose of a first recombinant herpesvirus (e.g., a recombinant HSV or a recombinant VZV) provided herein and a second recombinant herpesvirus (e.g., a recombinant CMV) provided herein can be delivered by a sustained release formulation. In some cases, a first recombinant herpesvirus and a second recombinant herpesvirus provided herein can be delivered in combination with one or more pharmacological agents that facilitate viral replication and spread within cancer cells, or agents that protect non-cancer cells from viral toxicity. Examples of such agents are described elsewhere (Alvarez-Breckenridge et al., Chem. Rev., 109 (7): 3125-40 (2009)).

The course of therapy with first and second recombinant herpesviruses provided herein can be monitored by evaluating changes in clinical symptoms or by direct monitoring of the number of cancer cells or size of a tumor. For a solid tumor, the effectiveness of virus treatment can be assessed by measuring the size or weight of the tumor before and after treatment. Tumor size can be measured either directly (e.g., using calipers), or by using imaging techniques (e.g., X-ray, magnetic resonance imaging, or computerized tomography) or from the assessment of non-imaging optical data (e.g., spectral data). The effectiveness of viral treatment also can be assessed by monitoring the levels of a cancer specific antigen. Cancer specific antigens include, for example, carcinoembryonic antigen (CEA), prostate specific antigen (PSA), prostatic acid phosphatase (PAP), CA 125, alpha-fetoprotein (AFP), carbohydrate antigen 15-3, and carbohydrate antigen 19-4.

In some cases, one or more recombinant herpesviruses described herein (e.g., a recombinant HSV having oncolytic anti-cancer activity, a recombinant VZV having oncolytic anti-cancer activity, a recombinant CMV having oncolytic anti-cancer activity, a combination of a recombinant HSV having oncolytic anti-cancer activity and a recombinant CMV having oncolytic anti-cancer activity, or a combination of a recombinant VZV having oncolytic anti-cancer activity and a recombinant CMV having oncolytic anti-cancer activity) can be formulated into a composition (e.g., a pharmaceutical composition) for administration to a mammal (e.g., a mammal having, or at risk of having, cancer). For example, one or more recombinant herpesviruses described herein can be formulated into a pharmaceutically acceptable composition for administration to a mammal having, or at risk of having, cancer. In some cases, one or more recombinant herpesviruses can be formulated together with one or more pharmaceutically acceptable carriers (additives) and/or diluents. A pharmaceutical composition can be formulated for administration in solid or liquid form including, without limitation, sterile solutions, suspensions, sustained-release formulations, tablets, capsules, pills, powders, and granules. Pharmaceutically acceptable carriers, fillers, and vehicles that may be used in a pharmaceutical composition described herein include, without limitation, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

In some cases, methods described herein also can include administering to a mammal (e.g., a mammal having cancer) one or more additional agents used to treat a cancer. The one or more additional agents used to treat a cancer can include any appropriate cancer treatment. In some cases, a cancer treatment can include surgery. In some cases, a cancer treatment can include radiation therapy. In some cases, a cancer treatment can include administration of a pharmacotherapy such as a chemotherapy, hormone therapy, targeted therapy, and/or cytotoxic therapy. For example, a mammal having cancer can be administered a first recombinant herpesvirus described herein (e.g., a recombinant HSV having oncolytic anti-cancer activity) and a second recombinant herpesvirus (e.g., a recombinant CMV having oncolytic anti-cancer activity) and administered one or more additional agents used to treat a cancer. For example, a mammal having cancer can be administered a first recombinant herpesvirus described herein (e.g., a recombinant VZV having oncolytic anti-cancer activity) and a second recombinant herpesvirus (e.g., a recombinant CMV having oncolytic anti-cancer activity) and administered one or more additional agents used to treat a cancer. In cases where a mammal having cancer is treated with first and second recombinant herpesviruses described herein and is treated with one or more additional agents used to treat a cancer, the additional agents used to treat a cancer can be administered at the same time or independently. For example, first and second recombinant herpesviruses described herein and one or more additional agents used to treat a cancer can be formulated together to form a single composition. In some cases, first and second recombinant herpesviruses described herein can be administered first, and the one or more additional agents used to treat a cancer administered second, or vice versa.

The invention will be further described in the following examples, which do not limit the scope of the invention described in the claims.

### EXAMPLES

Herpesviruses are a large group of nuclear-replicating (such that viral DNA is replicated and transcribed within nucleus) DNA viruses that share a common virion structure: a large, monopartite, double stranded linear DNA genome encoding 70-200 genes encapsulated within an icosahedral capsid, which is wrapped in a tegument layer and a lipid bilayer envelope (Dai and Zhou, Science 2018, 360 (6384): eaao7298). Herpesvirus infection is a powerful trigger to the initiation of rapid innate immune responses and subsequent antigen presentation, recruitment of adaptive immune cells and formation of memory cells (White et al., Immunol Rev 2012, 245 (1): 189-208). Another advantage of herpesvirus as an oncolytic agent is its large capacity for inserting therapeutic transgenes, which brings a variety of options to combine virotherapy with different therapies. Among the nine human herpesviruses, HSV-1 has been widely investigated, and in 2015 the FDA approved the first oncolytic recombinant HSV-1 (T-VEC, also known as OncoVEXGM-CSF) to treat melanoma. It is believed that the mechanism for T-VEC's antitumor efficacy includes infecting and killing of tumor cells, followed by release of tumor associated antigens that are then presented to adaptive immune cells, and establishment of systematic anti-tumor immunity (Conry et al., Hum Vaccine Immunother 2018, 14 (4): 839-846). A Phase 1b clinical trial confirmed that T-VEC treatment promoted both intratumoral T cell infiltration and anti-PD-1 immunotherapy (Ribas et al., Cell 2017, 170 (6): 1109-1119.e10). However, the detailed anti-tumor mechanism of HSV and all oncolytic viruses still is not well characterized.

In general, the methods and materials described herein include the use of viruses with attenuated virulence, while utilizing or enhancing the abilities of the viruses to induce differential immune responses and facilitate virus-mediated oncolytic immune therapy. In some cases, a virus can have a deletion of ICP47 and/or ICP34.5 (it is noted that T-VEC carries both deletions). ICP47 is a HSV encoded specific TAP complex inhibitor, the expression of which can block the activation of MHC1 mediated cytotoxic T and natural killer (NK) cell activation. The removal of ICP47 can partially restore viral/tumor antigen presentation, which can contribute to oncolytic efficacy (see, e.g., Liu et al., Gene Ther 2003, 10 (4): 292-303). ICP34.5 is a multifunctional virus encoded host regulator that promotes virus replication and survival through three distinct pathways (Wilcox and Longnecker, PLOS Pathog 2016, 12 (3): e1005449). The Beclin-1 binding domain of ICP34.5 inhibits host autophagy, which is likely implicated in host antigen processing and presentation. The TBK1 binding domain of ICP34.5 inhibits activation of the interferon pathway that is important for dendritic cell activation. The PPla and eIF2a binding domains of ICP34.5 are responsible for reversal of the host protein synthesis shut-off response, which is important for virus replication within infected cells (Wilcox and Longnecker, supra).

HSV-1, HCMV, and VZV present differential receptor avidities and cell tropisms when infecting a tissue, and they induce both similar and differential immune-responses post infection. 3-O-Sulfated Heparan Sulfate (3-OS—HS), herpesvirus entry mediator (HVEM), and Nectin1 have been found to serve as entry receptors for HSV-1, while Toll-like receptor (TLR), epidermal growth factor receptor (EGFR), and a specific subgroup of integrins can be used by CMV as entry receptors (Krummenacher et al., Adv Exp Med Biol 2013; 790:178-179). VZV uses heparan sulfate proteoglycan (HSPG) as an attachment receptor, but its entry receptors are still under investigated (Krummenacher et al., supra). HSV-1, HCMV, and VZV all induce strong NK cell and T cell immunity, but differential gamma-delta T cell responses.

VZV, HSV-1 and HSV-2 are the only three human alpha herpesviruses, and many structural and functional homologs have been identified between VZV and HSV (Zhang et al., PLOS Pathog 2010, 6: e1000971). During primary infection, VZV typically infects tonsil T cells, where new virions are synthesized and transmitted, and the infected T cells (especially infected cells with activation and skin-homing markers) are used by VZV as vehicles to spread systematically to distinct body skin sites (Arvin et al., Curr Top Microbiol Immunol 2010, 342:189-209). The natural tropism to infect skin (e.g., keratinocyte, melanocyte, and hair follicle cells) and T cells suggests that VZV is useful for treating T cell lymphoma and melanoma, or as a helper virus to enhance the oncolytic efficacy of HSV and CMV.

The utility of CMV as an oncolytic virus arises from its ability to target tumor-associated myeloid cells to change the immune-suppressive tumor microenvironment. However, safety concerns exist since wild type HCMV is prone to establish persistent or latent infection in vivo. More than 15 kb of the wild type HCMV genome encodes viral products that directly interact with and inhibit the function of NK cells and T cells (Wang et al., J Virol 2013, 87 (11): 6359-6376). These viral products or RNAs help the slow-replicating HCMV to survive and establish persistent infection in vivo. The nearly 20 kb NK and T cell inhibitory region is lost in many lab-adapted strains, which makes virus infected cells vulnerable under the attack of immune cells and therefore may prevent the virus from systematic spreading or latency in vivo (Gavin et al., Med Microbiol Immunol 2015, 204 (3): 273-284).

Lab adapted CMV stains have been used to develop vaccines (Anderholm et al., Drugs 2016, 76 (17): 1625-1645). As described herein, lab adapted AD169-derived viruses were used to target glioma and the tumor microenvironment. Since AD169 carries a mutation within the UL131 ORF that leads to a damaged gH/gL/UL128-131 glycoprotein complex, the function of this complex responsible for virus non-fibroblast entry was repaired by replacing the UL131 like sequence in AD169 with the functional UL131 ORF from the Merlin strain. In vivo studies showed that both the AD169 virus and the UL131 repaired virus presented potent oncolytic efficacy in the U251 model. In addition, viruses with the US1-US11 region deleted promoted establishment of virus infection within tumors but not a significant improvement for efficacy, likely due to the impaired activation of host NK cells when the MHC-I pathway was not inhibited by US1-US11 (Zou et al., J Immunol 2005, 174 (5): 3098-104). In addition viruses expressing wild type or EGFR-retargeted measles F/H glycoproteins or the suicide CD::UPRT fusion protein were tested, revealing that EGFR retargeted measles F/H enhanced virus spreading within tumors and improved anti-tumor efficacy.

Using the natural tropisms and replication characteristics of the three human herpesviruses, HSV-1, CMV, and VZV, the virus templets were designed by adding reporter genes, mutating viral genes, enhancing virus spread and immunogenic cell death, and inserting boosting transgenes. The complementary viral tropisms and characteristics between alpha (e.g., HSV and VZV) and beta (e.g., CMV) herpesviruses were combined to increase oncolytic effects. The viruses were used as monotherapies or combination therapies in in vivo tumor models to evaluate oncolytic properties and mechanisms.

### Example 1-Construction of Recombinant Herpesviruses

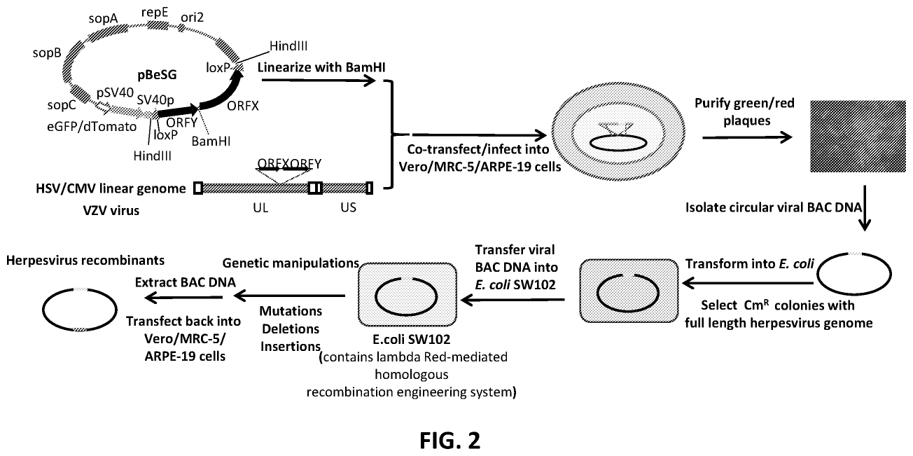

The herpesvirus construction work utilized an established bacterial artificial chromosome (BAC) homology recombination system that is highly efficient for introducing mutations, deletions, and insertions into the herpesvirus genome within bacterial cells (see, e.g., Paredes and Yu, Curr Protoc Microbiol 2012, Chapter 14: Unit 14E.4). Purified virus can be obtained within weeks using this platform. In the present work, based on the bacterial artificial chromosome (BAC) vector based HR technique, four herpesviral BAC viruses (HSV-1 KOS-BAC, HCMV AD169-BAC, mCMV Smith-BAC, and VZV vOka-BAC) were constructed using the method depicted in FIG. 2. Circular viral BAC DNA was isolated and transformed into E. coli cells, within which the BAC vector supported replication of the whole KOS-BAC DNA. The sopA-C elements of the BAC vector kept the plasmid at a low copy status and also kept the whole sequence stable. Genetic manipulations of the KOS genome were achieved in SW102 bacterial cells (which contain a lambda Red-mediated HR recombineering system), and pure BAC DNA was extracted from a single colony and then transfected into mammalian cells to reconstitute virus with a unified genotype.

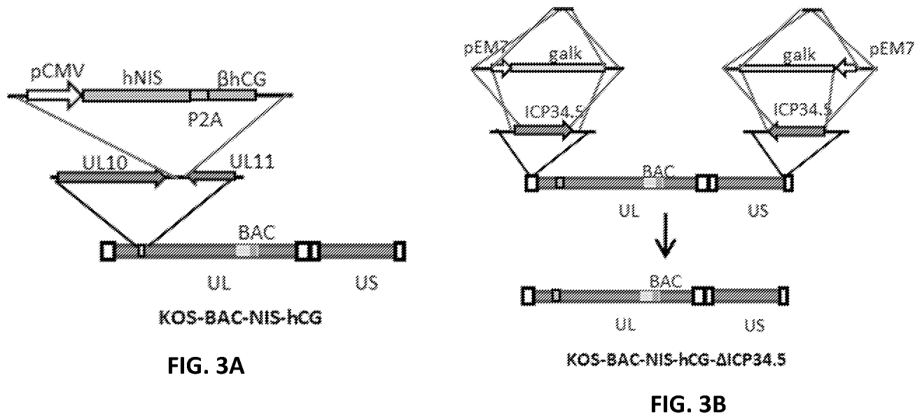

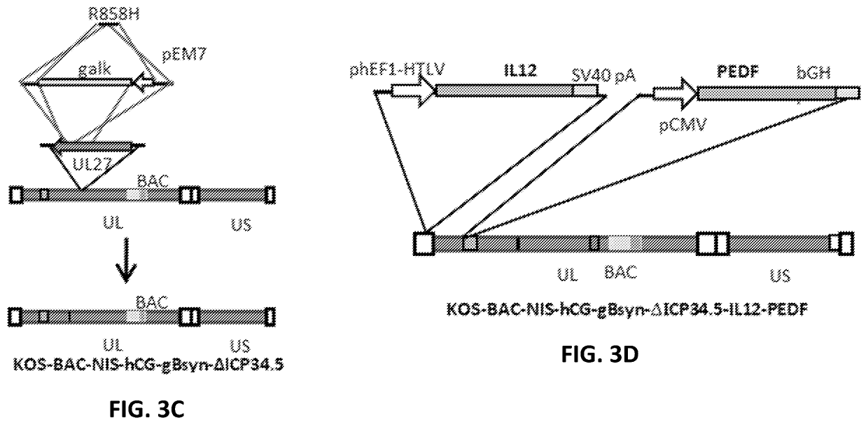

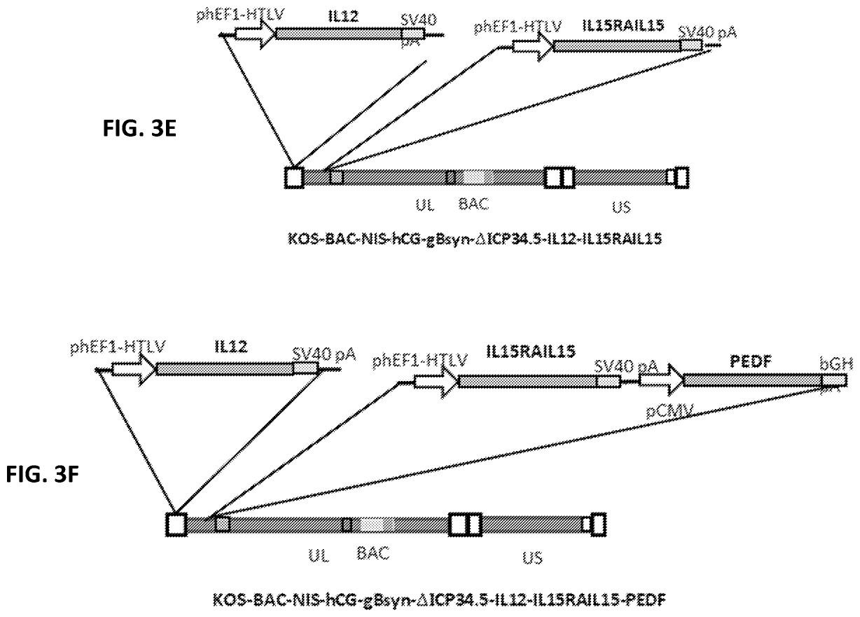

Based on the BAC-HR technical platform, a variety of HSV-1, HCMV, mCMV, and VZV recombinants were generated. The genome structures of those BAC constructs are shown in FIGS. 3A-3F, 4A-4E, and 5.

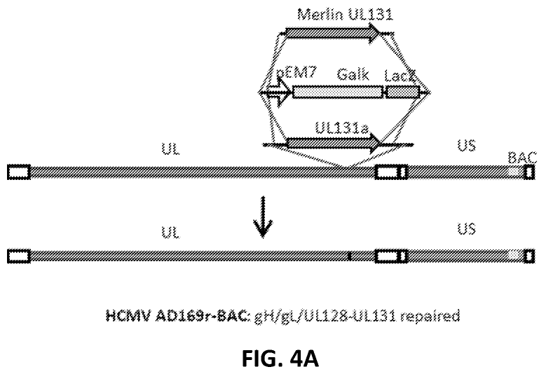

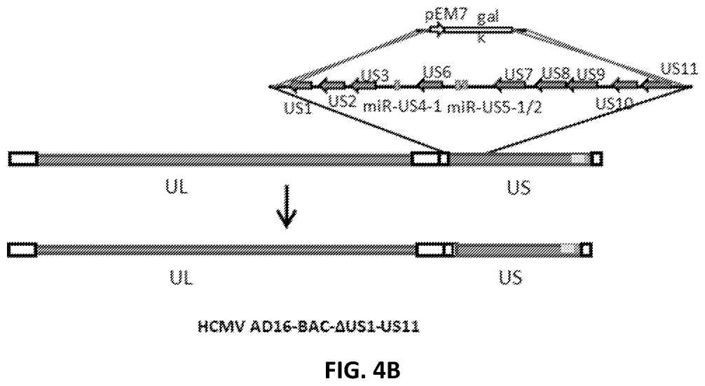

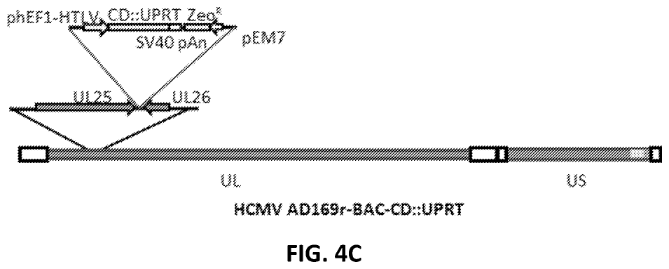

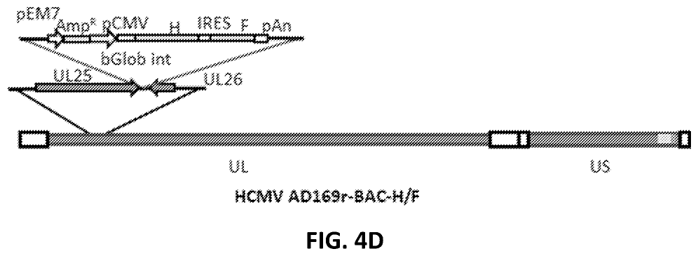

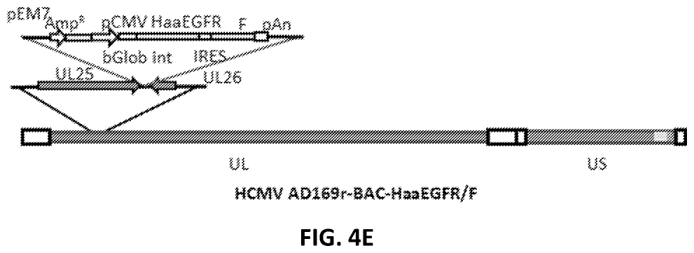

For HCMV, the lab-adapted strain AD169 was chosen as the templet virus. The AD169 genome lacks the UL1-UL18 and UL/b′ sequences; the protein and RNA products from those two regions inhibit NK and cytotoxic T cell activation, so AD169 derived viruses have substantially lost the ability to resist cytotoxic immune cell attack in vivo and would be safe as oncolytic viruses. Based on the BAC HR technical platform, a variety of AD169 recombinants were generated, including AD169-BAC (FIG. 4A), AD169-BAC-AUS1-US11 (FIG. 4B), AD169-BAC-CD::UPRT (FIG. 4C), AD169-BAC—H-IRES-F (FIG. 4D), and AD169-BAC-HaaEGFR-IRES-F (FIG. 4E), as well as AD169r-BAC, AD169-BACr-AUS1-US11, AD169-BACr-CD::UPRT, AD169-BACr—H-IRES-F, and AD169-BACr-HaaEGFR-IRES-F. AD169-BAC-CD::UPRT included a uracil phosphoribosyl transferase (UPRT) coding sequence fused to the cytosine deaminase (CD) gene (CD::UPRT). CD converts 5-fluorocytosine (5-FC) to 5-fluorouracil (5-FU) and UPRT converts 5-FU to 5-FUMP, an irreversible inhibitor of thymidylate synthase. This is 100 times more efficient than the CD gene alone and provide a greater bystander effect. CD::UPRT was included to enhance immunogenic cell death, hemagglutinin/fusion coding sequences (H/Fs) also were included in some of the constructs to enhance cell fusion-mediated spread of CMV and to enhance immunogenic cell death, while EGFR (which is highly expressed on glioma cells) retargeted H/Fs to improve the selectivity of cell fusion.

The viruses were reconstituted in MRC-5 cells. Since AD169 carries a mutation within the UL131 ORF that leads to a damaged gH/gL/UL128-131 glycoprotein complex, the function of this complex responsible for virus non-fibroblast entry was initially repaired by replacing the UL131-like sequence in AD169 with the functional UL131 ORF from Merlin strain. As noted above, the insertion of CD::UPRT was made to enhance immunogenic cell death, and H/Fs were inserted to enhance the cell fusion-mediated spread of CMV and to enhance immunogenic cell death, and EGFR to retarget H/F and increase the selectivity of cell fusion. H/Fs mediated cell fusion was verified in U87 cells.

Compared to other alpha herpesviruses, VZV is a strictly cell-associated virus and does not grow to a high titer, and infectious virions are barely detectable in supernatants of VZV infected cells. Potential advantages of using VZV as oncolytic virus include (1) its potent capability to recruit leukocytes (especially T cells) through cytokines and the virus-encoded chemokine binding protein, gC; (2) its capability to recruit NK cell immunity; and (3) its natural tropism to T cell and skin cells. Using the Oka varicella vaccine (Merck), the BAC virus vOka-BAC was generated. As an alpha-herpesvirus, VZV does not encode homologs of HSV ICP47 (a MHC pathway inhibitor) or ICP34.5 (a host regulator). In addition, the mRNAase activity of ORF17 is less potent than HSV UL41. Further attenuation to this vaccine-derived virus (e.g., by deletion of viral genes or insertion of foreign genes) may attenuate virus replication and reduce its oncolytic value.

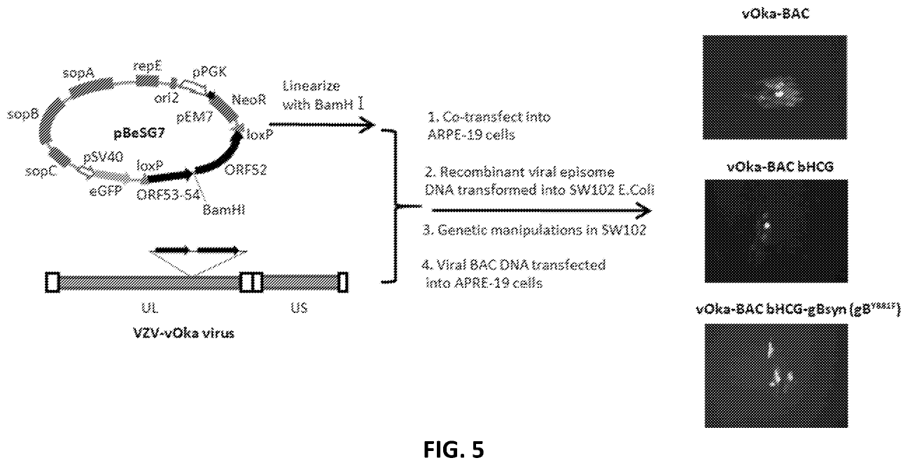

Starting with the Merck live attenuated vOka strain, vaccine powder was added into epithelial cells and a BAC virus was generated as illustrated in FIG. 5. Recombinant viral constructs carrying NIS, or beta-hCG, or both NIS and beta-hCG were obtained. It was observed that the recombinant VZVs containing NIS or containing both reporters were difficult to rescue, while the vOka-BAC-hCG virus grew like the WT virus (FIG. 5). This suggested that NIS expression interfered with VZV replication. Based on the vOka-BAC-bHCG virus, the gB hyperfusogenic mutation Y881F was added; this rescued the recombinant virus vOka-BAC-bHCG-gBsyn.

### Example 2-In Vitro Characterization of Virus Growth and Reporter Expression

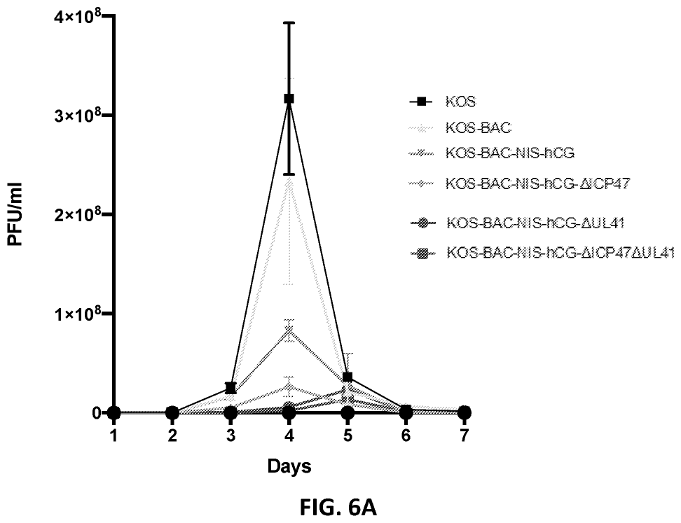

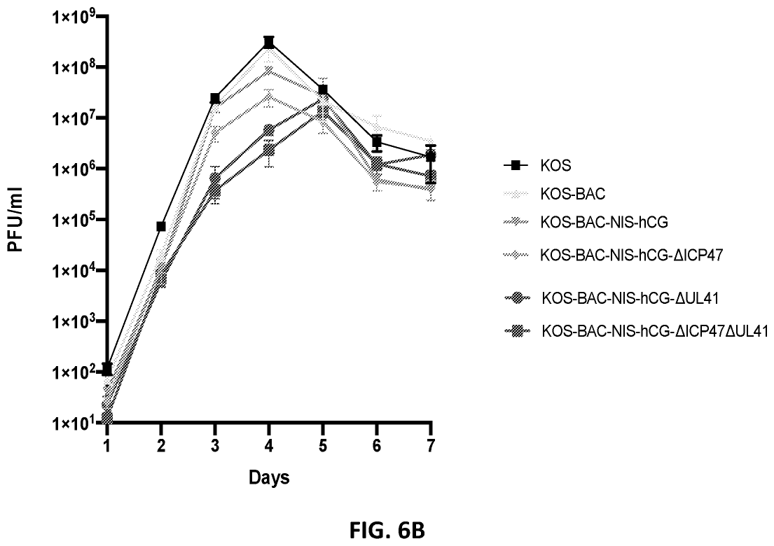

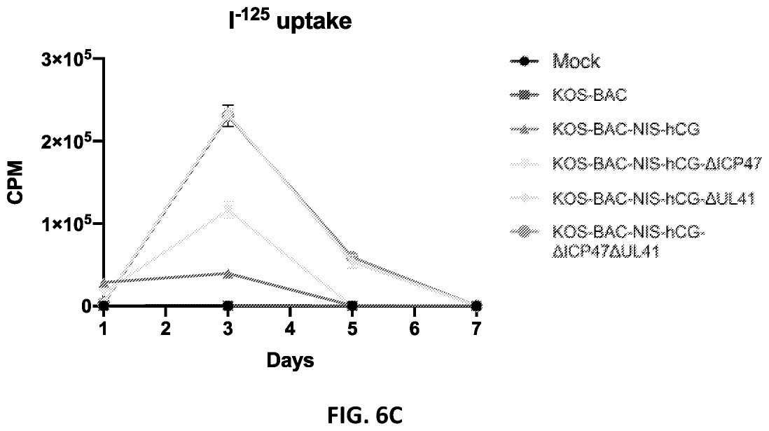

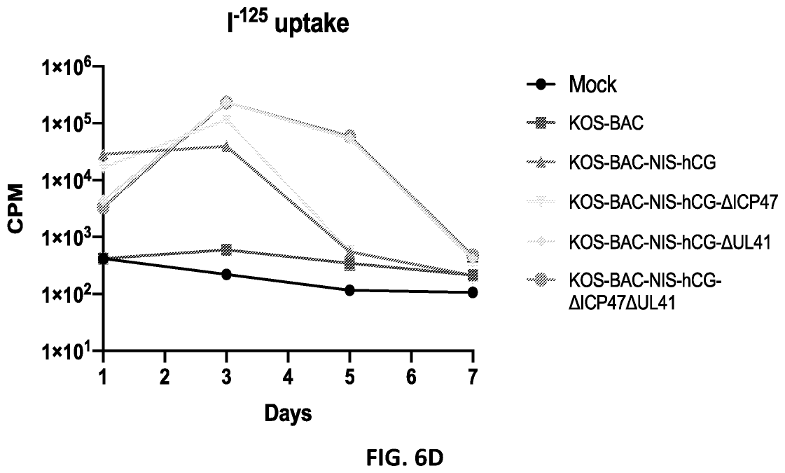

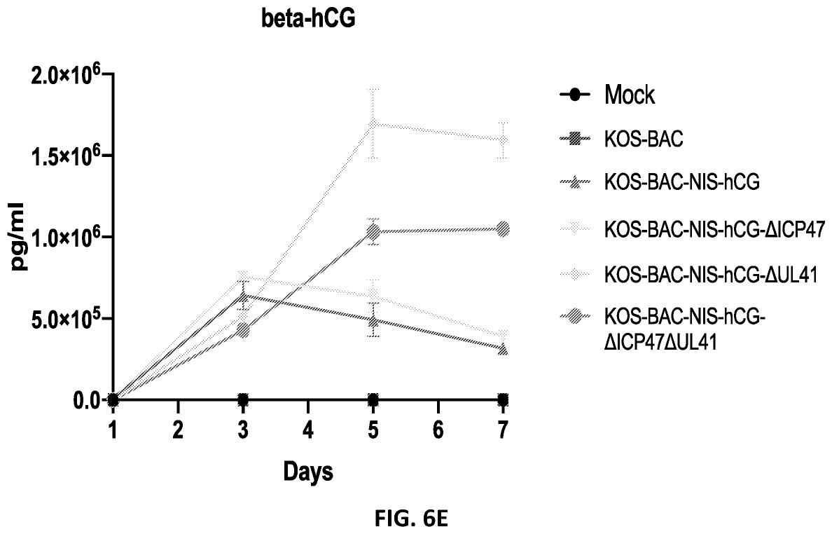

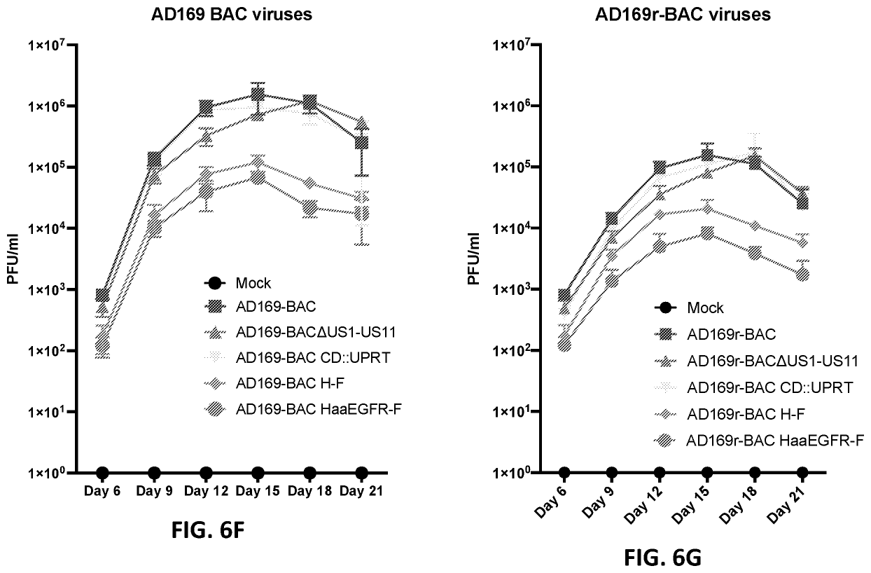

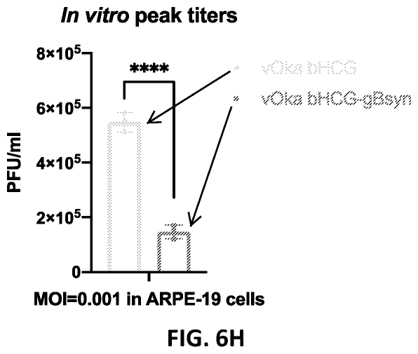

After reconstitution of the HSV-1 KOS derived viruses, their growth kinetics were analyzed in Vero cells. As shown in FIG. 6A, all of the genetic manipulations (insertion of the BAC vector, addition of the NIS-HCG cassette, and knock out of ICP47) attenuated virulence, and a significant drop in peak titer was observed after the insertion and expression of NIS-HCG. The deletion of UL41, either alone or together with ICP47, further significantly attenuated virus replication at 4 dpi (FIG. 6B), and even the peak titers of AUL41 viruses shifted from 4 dpi to 5 dpi. It was noted that the attenuation caused by AUL41 may be amplified in vivo due to the pressure from immune system. Activity of the two reporters (hNIS and beta-hCG) was then measured in Vero cells. As shown in the curves plotted for NIS expression (FIGS. 6C and 6D) and hCG secretion (FIG. 6E), AUL41 significantly increased the peak level of both reporters, due to the absence of the mRNA-degrading UL41 (vhs). In further studies, growth curves for the HCMV AD169-BAC derived viruses were determined in MRC-5 cells (FIGS. 6F and 6G), and VZV derived viruses were evaluated in ARPE-19 cells. The latter studies revealed that the VZV gB hyperfusogenic mutation, Y881F, attenuated virus growth in vitro (FIG. 6H).

### Example 3-In Vivo Studies

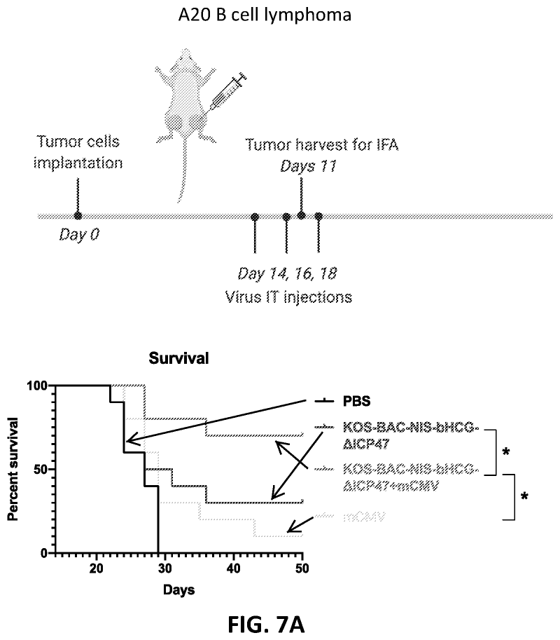

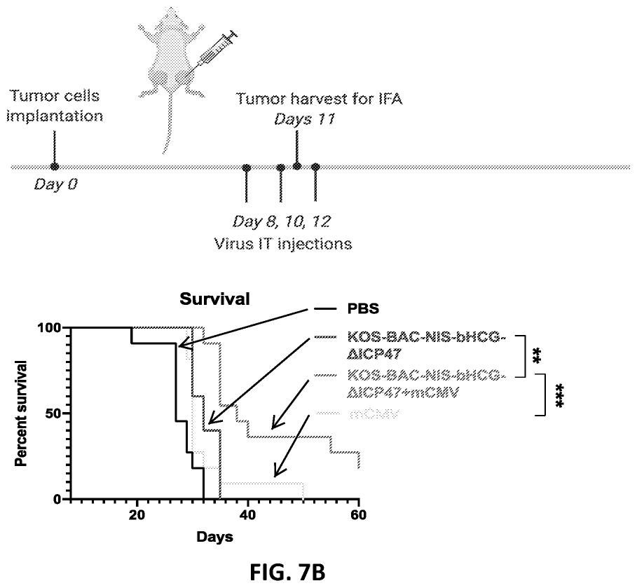

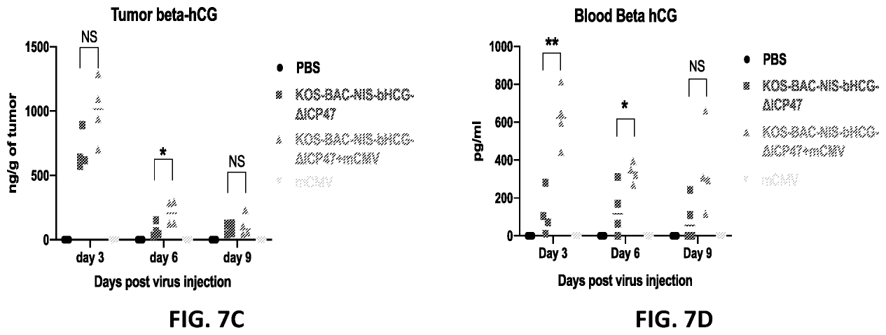

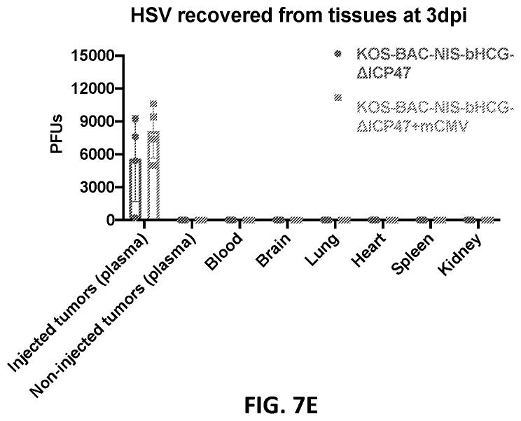

Classical tumor cell line models for HSV oncolytic virotherapy include the A20 B cell lymphoma model and the EMT6 breast cancer syngeneic model, both of which have tumorigenic abilities in immune-competent mice. These lines were selected for in vivo studies. Initial studies showed that the combination of HSV-1KOS-BACΔICP47 and mCMV had substantially increased anti-tumor effects, as compared to HSV or CMV monotherapy, in syngeneic A20 and EMT6 tumor models (FIGS. 7A and 7B). Co-injection of mCMV also slightly increased the expression of the HSV beta-hCG reporter gene (FIGS. 7C and 7D), and virus was not detected in tissues beyond the injected tumors (FIG. 7E).

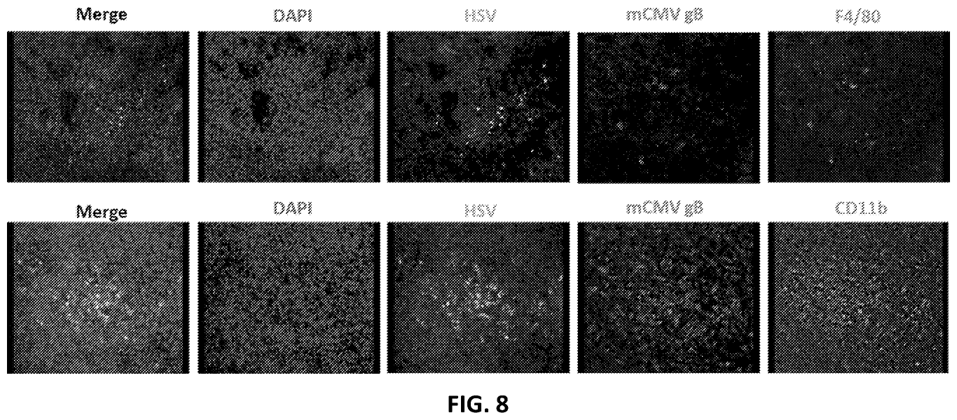

Immunofluorescence analysis was conducted for HSV plus mCMV co-injected EMT6 tumors. HSV plus mCMV infected cells were stained with Alexa fluor-488 conjugated anti-GFP and rabbit anti-mCMV gB. Macrophage markers F4/80 and CD11b were co-stained. These studies showed that HSV-1 and mCMV infected different cell types within the EMT6 tumors, and that mCMV targeted tumor-associated macrophages (FIG. 8).

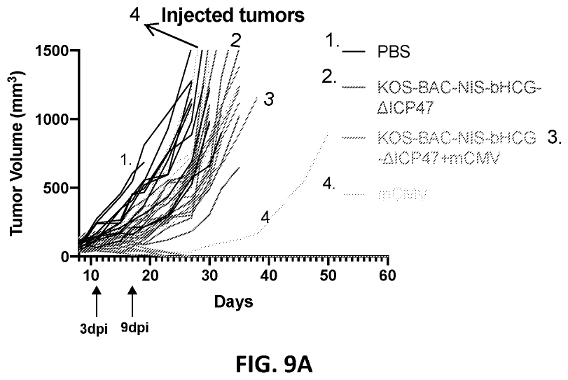

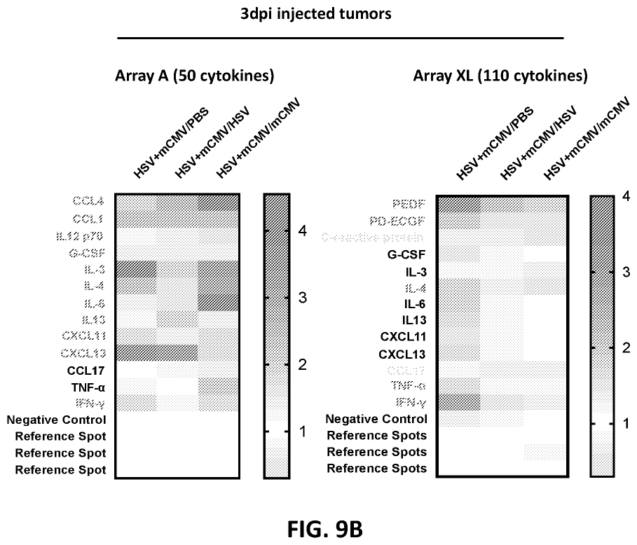

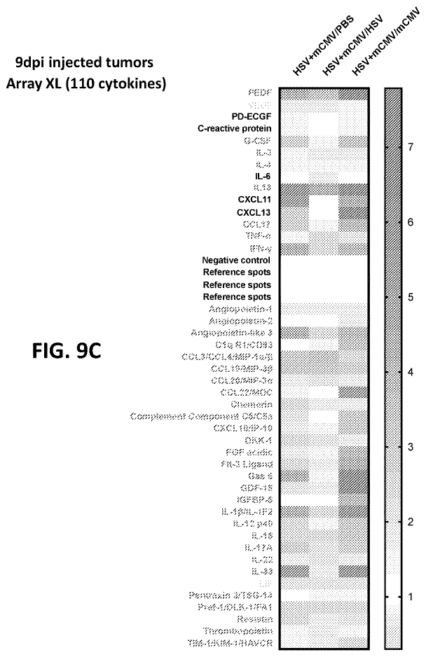

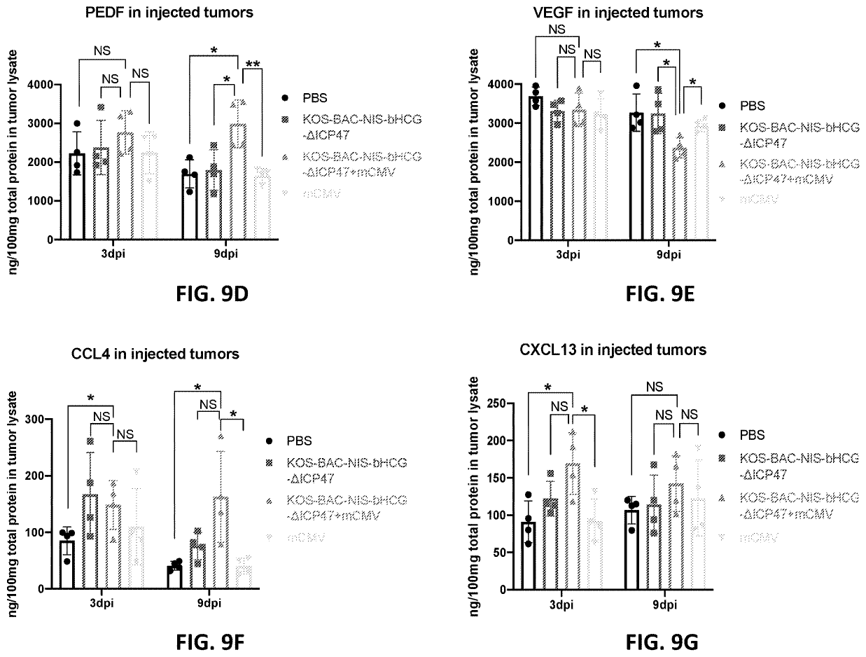

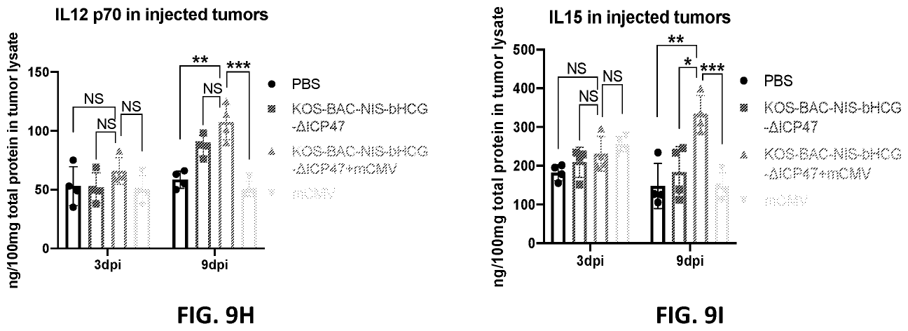

Cytokine profiling for the injected tumors (FIG. 9A) demonstrated that more cytokines were upregulated in the HSV plus mCMV injected tumors at 9 dpi (FIG. 9C) than at 3 dpi (FIG. 9B). Five of these upregulated cytokines (pigment epithelium-derived factor (PEDF), CCL4, CXCL13, IL12, and IL15), along with VEGF, were further analyzed by ELISA (FIGS. 9D-9I). EDF, CCL4, CXCL13, IL12, and IL15 were selected as candidate therapeutic transgenes.

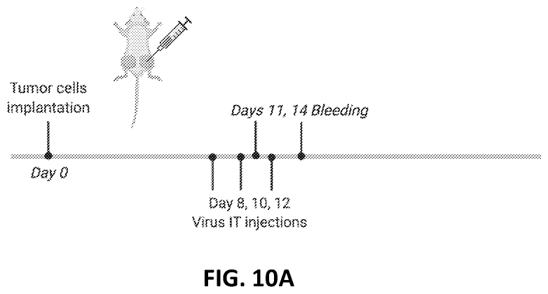

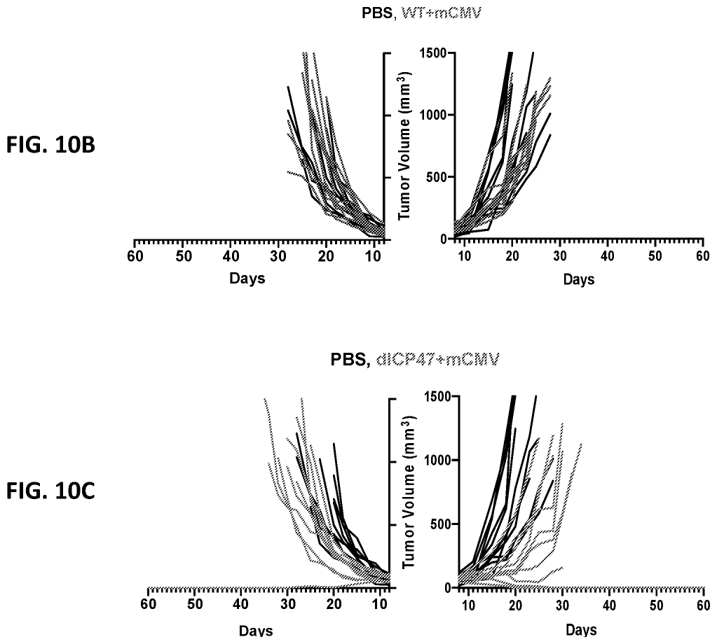

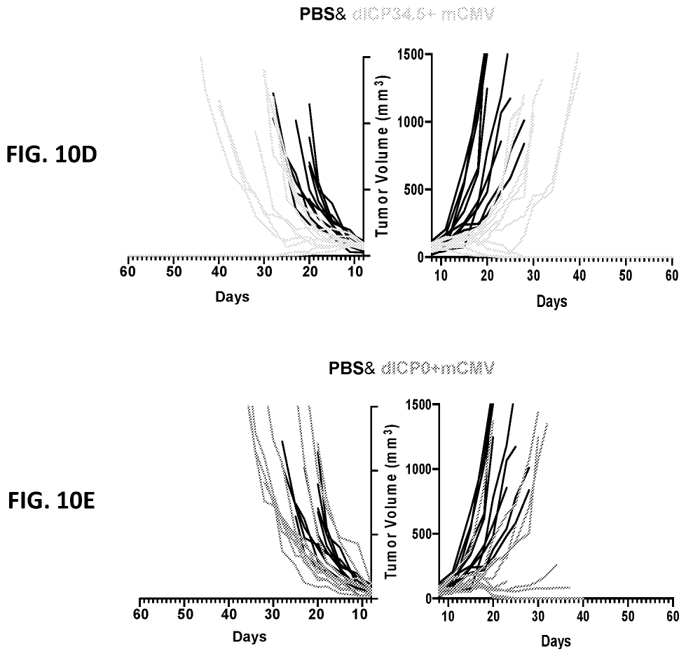

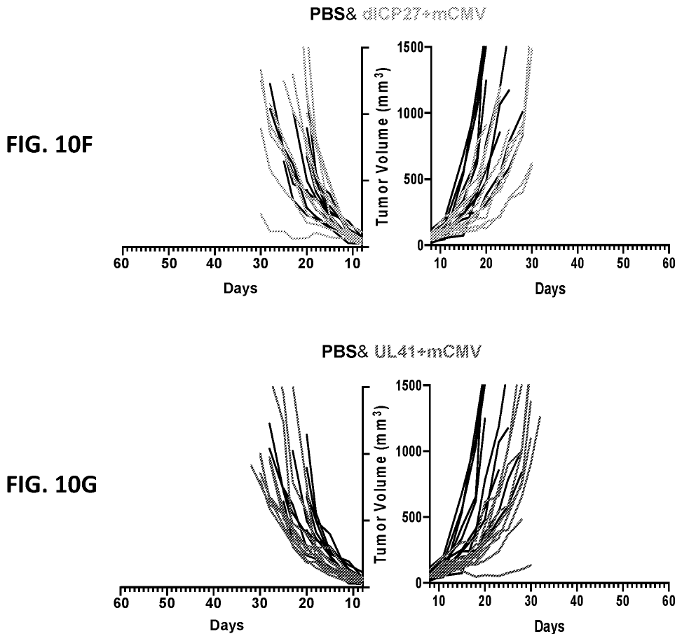

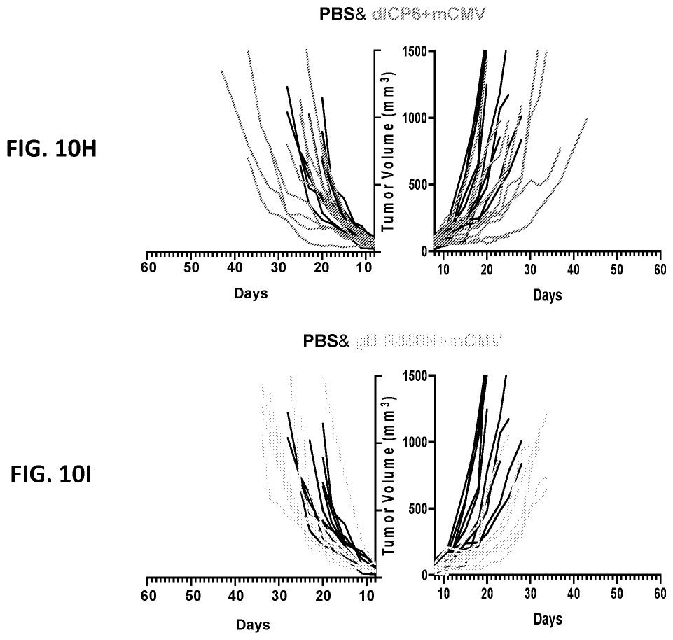

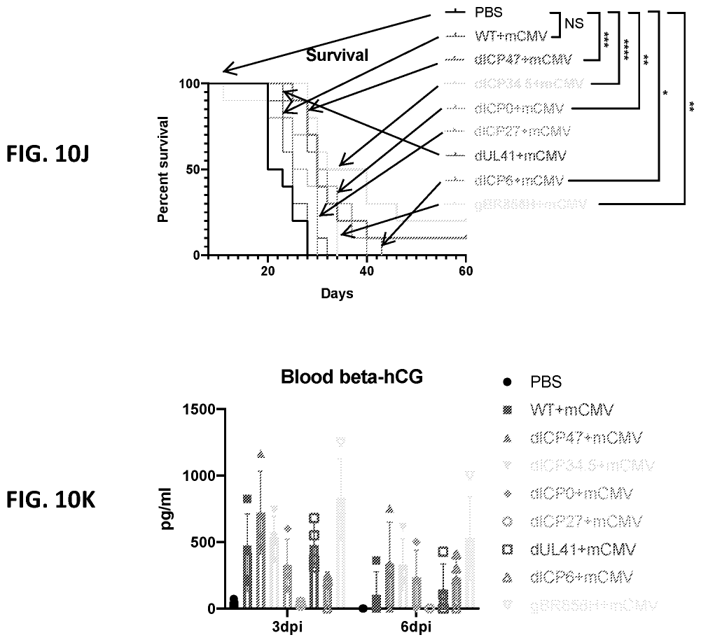

To obtain an optimized HSV-1 vector, HSV KOS-BAC-NIS-bHCG variants carrying deletion or mutation of different viral genes (ICP47, ICP34.5, ICPO, ICP27, ICP6, UL41, and gB) were generated and evaluated in vivo in the EMT6 model (FIGS. 10A-101). These studies demonstrated that the deletion of ICP34.5s (FIG. 10D) and the gBsyn (R858H) mutation (FIG. 10I) significantly improved efficacy as compared to WT virus (FIG. 10B) when combined with mCMV. Survival also was improved with deletion of ICP34.5s as well as UL41 (FIG. 10J). Measurement of blood beta-hCG levels (FIG. 10K) demonstrated that the gB hyperfusogenic mutation improved the virus' in vivo intratumoral spreading. These results further confirmed the conclusion that the combination of HSV/VZV plus CMV can significantly improve anti-tumor effects, and the vector KOS-BAC-NIS-hCG-gBsyn-ΔICP34.5s can be used to encode additional therapeutic transgenes and to combine with CMV.

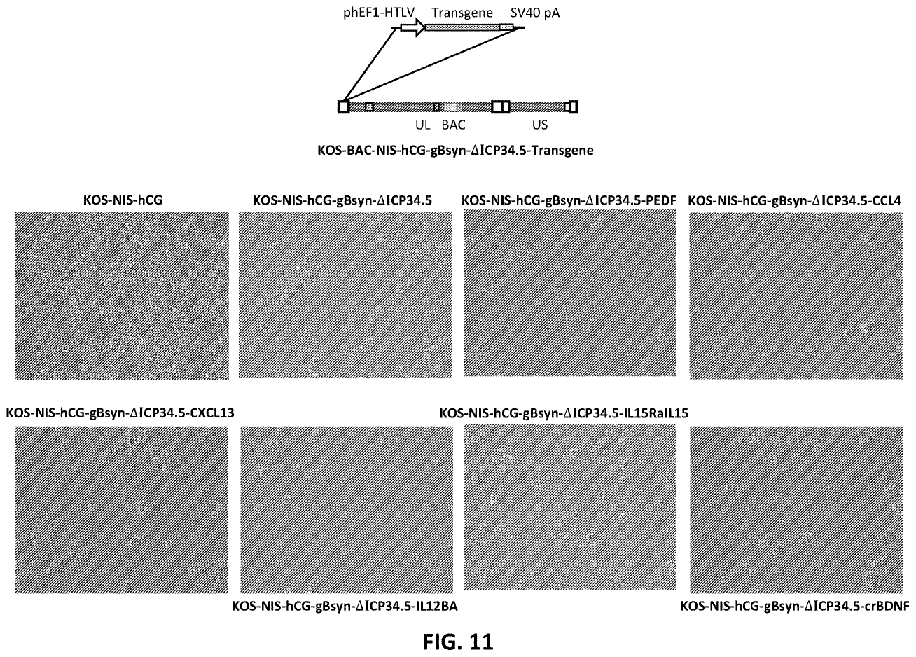

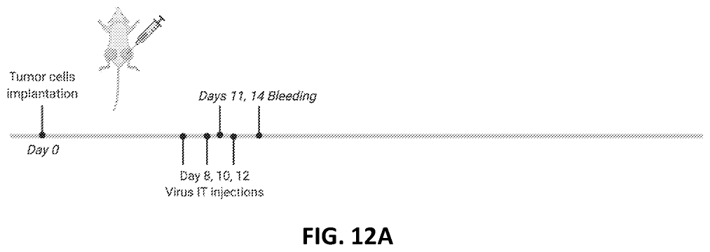

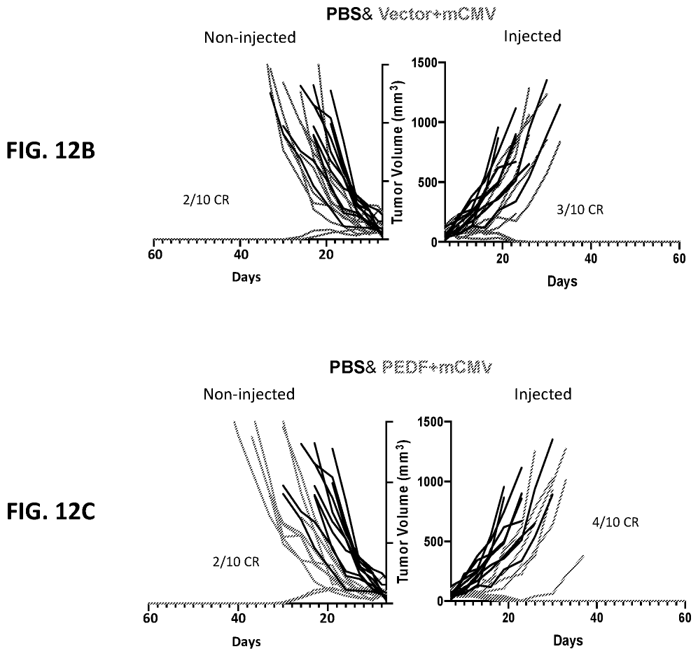

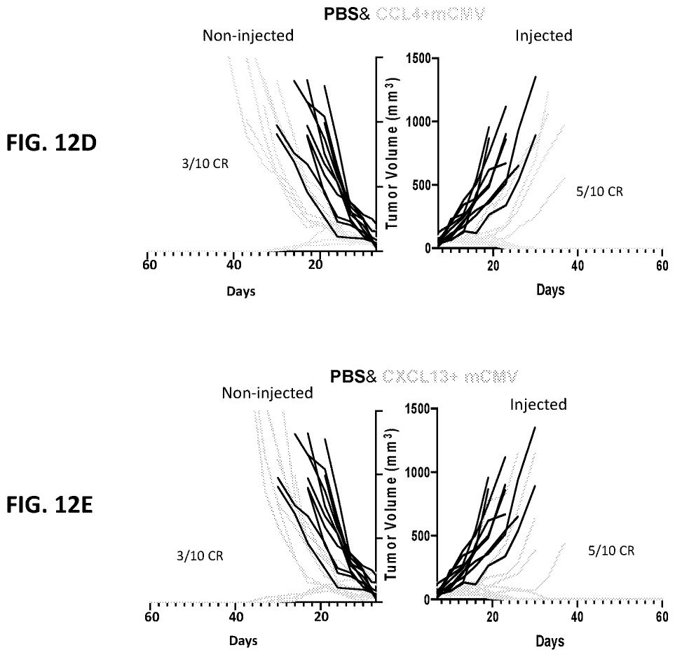

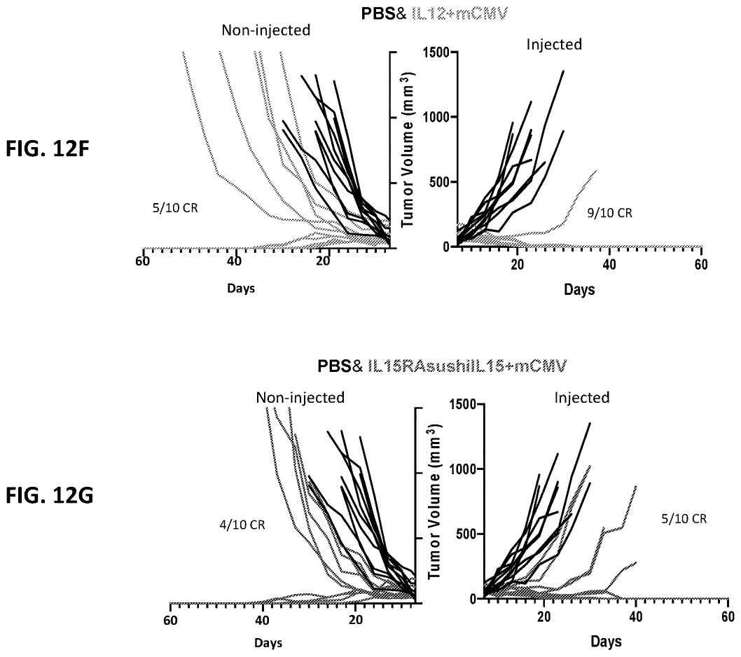

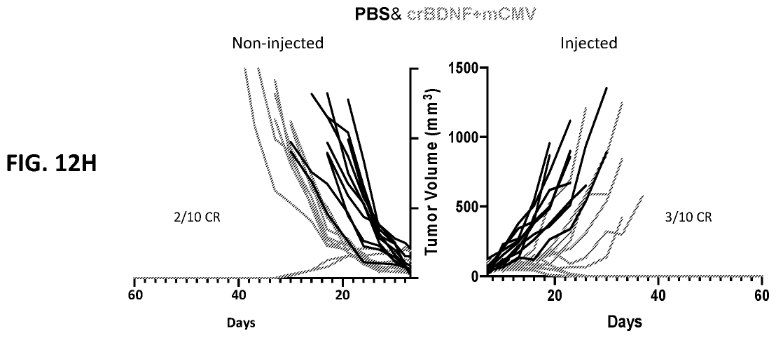

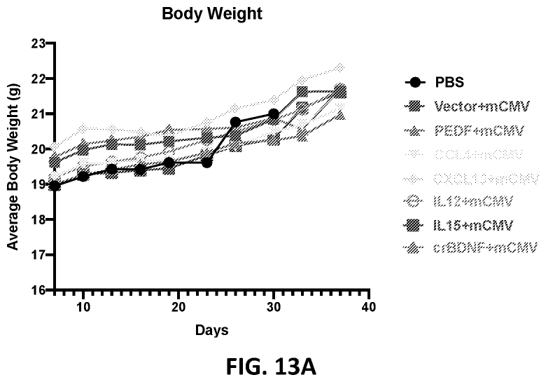

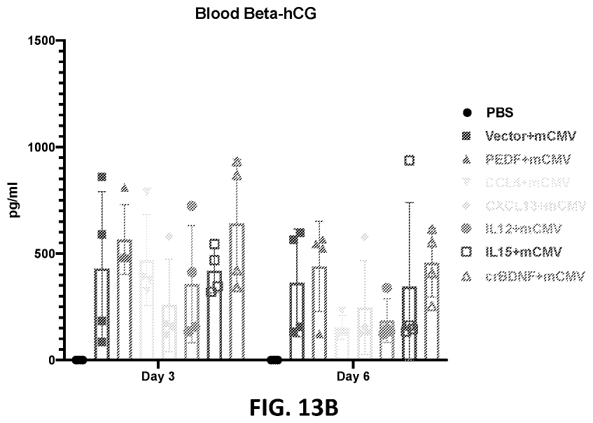

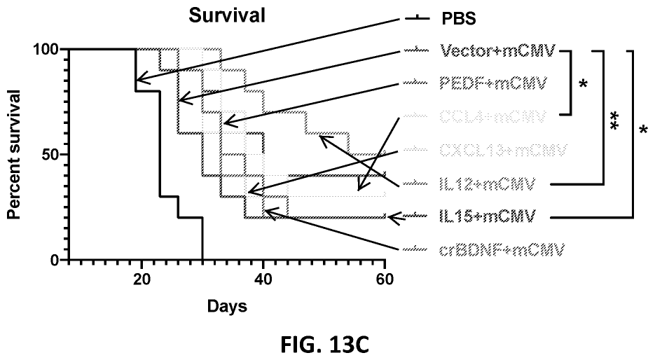

A number of different therapeutic transgenes were added into the HSV KOS-BAC-NIS-hCG-gBsyn-ΔICP34.5 construct. These included CXCL-13 (which is associated with pro-lymphoid neogenesis), CCL-4 (macrophage inflammatory protein-1, a chemoattractant for natural killer cells, monocytes and a variety of other immune cells), PEDF (an anti-angiogenic protein), IL12 (which promotes T cell and NK functions), IL15RAsushi-IL15 (which has pro-NK and T cell functions), and mutBDNF-mutNGF (pro-differentiation proteins) (FIG. 11). These transgenes may drive distinct immune-responses within tumors, which could significantly improve oncolytic effects. In vivo testing of the KOS-BAC-NIS-hCG-gBsyn-ΔICP34.5-Transgene combination with mCMV in the EMT6 model (FIG. 12A) revealed significant improvements in efficacy for all of the six cytokines coding sequences that were inserted as compared to PBS control (FIGS. 12C-12H and 13A-13C) or the Vector plus mCMV combination (FIG. 12B). Together, the results provided in FIGS. 12A-12H and 13A-13C demonstrated safety, virus replication in vivo, and improved efficacy.

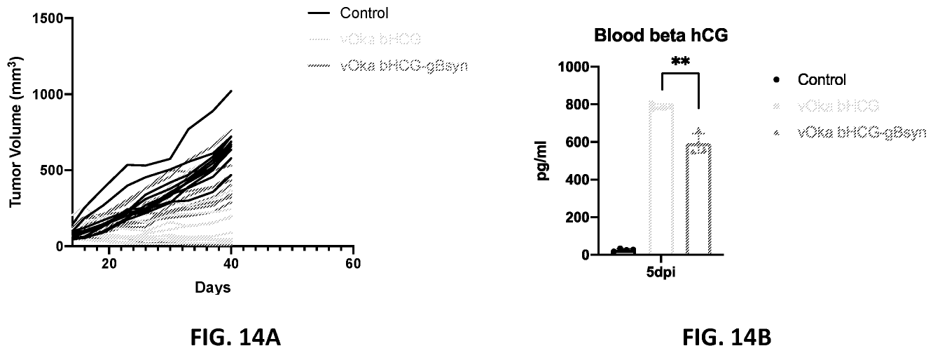

Reconstituted vOka-bHCG and vOka-bHCG-gBsyn viruses were tested in vivo using the human MeWo melanoma xenograft model. MeWo cells were implanted subcutaneously into the right flank of nude mice (5×10⁶ cells/mouse) at day 0, and at days 13, 15, and 17, 5×10⁵ thawed MeWo cells or 5×10⁵ thawed v-Oka infected MeWo cells were IT injected into the MeWo tumors. These studies showed that vOka-bHCG, without the hyperfusogenic gB mutation, was superior to the gB mutated virus for inhibiting tumor growth (FIG. 14A). In addition, blood beta-hCG levels indicated that the gB mutation hindered in vivo replication of the virus (FIG. 14B).

CMV is a slowly replicating virus and generally is not good at killing cancer cells, but it can be advantageous in that (1) it has tropism to infect endothelial and macrophages, which is different from the tropism of HSV-1 may confer utility as a modifier of the tumor microenvironment, and (2) it has the ability to induce strong NK, T, and B cell responses. Studies were conducted to determine whether combining HSV-1 therapy with CMV therapy would yield a significant increase in oncolytic effects.

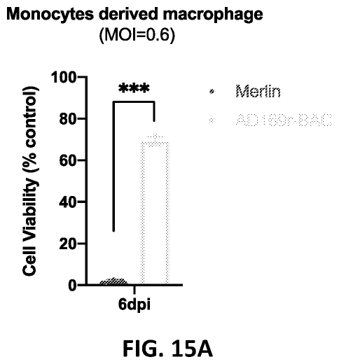

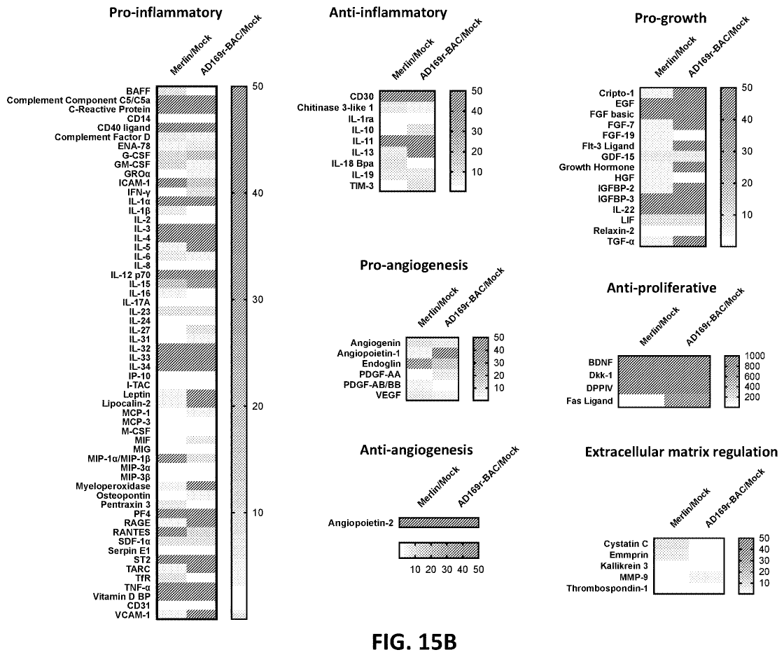

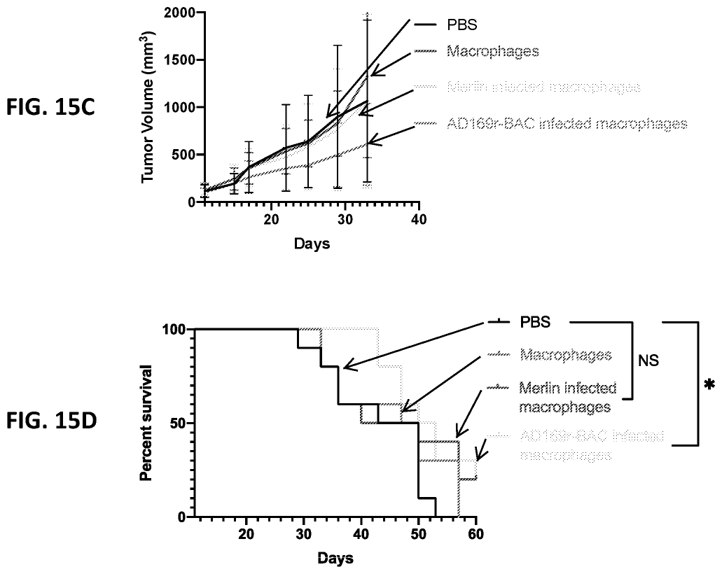

In these studies, infection of HCMV Merlin and AD169-rBAC was first characterized in monocyte-derived macrophages in vitro. Cytotoxicity analyses demonstrated that AD169r-BAC was significantly less cytotoxic than the wild type Merlin strain (FIG. 15A), but both strains substantially changed cytokine secretion in macrophages, driving increased expression of pro-inflammatory cytokines (FIG. 15B). The virus-infected macrophages also were injected into U251 xenograft tumors. Injection of AD169-rBAC infected macrophages more potently inhibited tumor growth than the wild type virus Merlin (FIG. 15C), suggesting the utility of VZV plus AD169r-BAC combinations. Both strains also resulted in a non-significant improvement in survival (FIG. 15D).

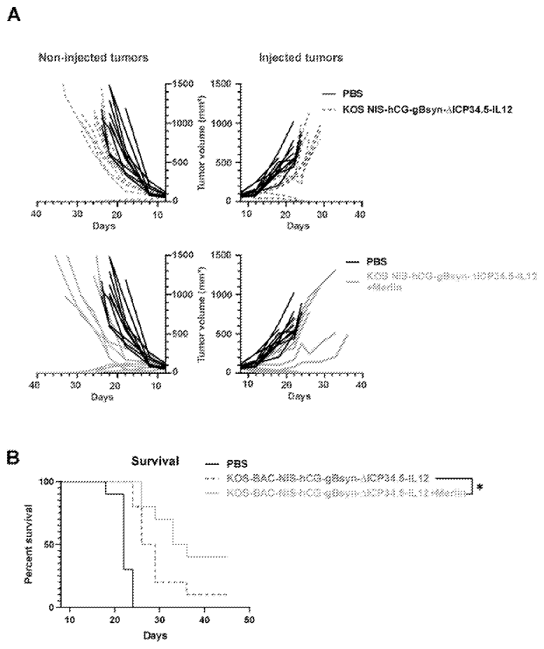

In the immunocompetent EMT6 model, co-delivery of HCMV Merlin strain with the HSV KOS-BAC-NIS-hCG-gBsyn-ΔICP34.5-IL12 elicited enhanced anti-tumor responses to both the treated and non-treated tumors (FIGS. 16A and 16B). In the combination therapy group, 40% of animals exhibited complete response and demonstrated long-term survival (FIG. 16B).

### Example 4-Additional HSV Vectors

HSV-1 vectors expressing IL12, PEDF, and IL15RA/IL15, as well as a vOka-bHCG-IL12 vector, are constructed and confirmed for efficacy in vitro and in vivo as described in Examples 2 and 3.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A composition comprising a first recombinant human herpesvirus and a second recombinant human herpesvirus, wherein said first and second recombinant human herpesviruses are from two different herpesvirus sub-families, and wherein, when said composition is administered to a mammal having cancer, said first recombinant human herpesvirus infects a cancer cell and said second recombinant human herpesvirus infects a stromal cell associated with said cancer, thereby reducing the number of cancer cells within said mammal.

2. The composition of claim 1, wherein said first recombinant human herpesvirus is a human alpha herpesvirus.

3. The composition of claim 2, wherein said human alpha herpesvirus is a recombinant human herpes-simplex virus (HSV) or a recombinant human varicella-zoster virus (VZV).

4. The composition of claim 2, wherein said human alpha herpesvirus is a recombinant human HSV.

5. The composition of claim 4, wherein said recombinant human HSV (a) lacks both copies of ICP34.5, (b) lacks functional ICP47, (c) contains nucleic acid encoding a mutant gB polypeptide having hyperfusogenic activity, or (d) contains a heterologous nucleic acid sequence encoding a therapeutic polypeptide.

6-9. (canceled)

10. The composition of claim 5, wherein said recombinant human HSV contains said heterologous nucleic acid sequence.

11. The composition of claim 10, wherein said therapeutic polypeptide is selected from the group consisting of pigment epithelium-derived factor (PEDF), CCL4, CXCL13, IL-12, IL-15, and brain-derived neurotropic factor (BDNF).

12-37. (canceled)

38. A method for treating a mammal having cancer, wherein said method comprises administering, to said mammal, a composition of comprising a first recombinant human herpesvirus and a second recombinant human herpesvirus, wherein said first and second recombinant human herpesviruses are from two different herpesvirus sub-families, and wherein, when said composition is administered to said mammal having cancer, said first recombinant human herpesvirus infects a cancer cell and said second recombinant human herpesvirus infects a stromal cell associated with said cancer, thereby reducing the number of cancer cells within said mammal.

39. The method of claim 38, wherein said mammal is a human.

40. The method of claim 38, wherein said method comprises administering, to said mammal, one or more therapies selected from the group consisting of radiation therapy, chemotherapy, and immunotherapy.

41. A method for treating a mammal having cancer, wherein said method comprises:
(a) administering, to said mammal, a first recombinant human herpesvirus, and
(b) administering, to said mammal, a second recombinant human herpesvirus,
wherein said first and second recombinant human herpesviruses are from two different herpesvirus sub-families, and wherein said first recombinant human herpesvirus infects a cancer cell of said mammal and said second recombinant human herpesvirus infects a stromal cell associated with said cancer, thereby reducing the number of cancer cells within said mammal.

42. The method of claim 41, wherein said mammal is a human.

43. The method of claim 41, wherein said first recombinant human herpesvirus is administered to said mammal before said second recombinant human herpesvirus.

44-45. (canceled)

46. The method of claim 41, wherein said method comprises administering, to said mammal, one or more therapies selected from the group consisting of radiation therapy, chemotherapy, and immunotherapy.

47. The method of claim 41, wherein said first recombinant human herpesvirus is a human alpha herpesvirus.

48. The method of claim 47, wherein said human alpha herpesvirus is a recombinant human HSV or a recombinant human VZV.

49. The method of claim 47, wherein said human alpha herpesvirus is a recombinant human HSV.

50. The method of claim 49, wherein said recombinant human HSV (a) lacks both copies of ICP34.5, (b) lacks functional ICP47, (c) contains nucleic acid encoding a mutant gB polypeptide having hyperfusogenic activity, or (d) contains a heterologous nucleic acid sequence encoding a therapeutic polypeptide.

51-54. (canceled)

55. The method of claim 50, wherein said recombinant human HSV contains said heterologous nucleic acid sequence encoding a therapeutic polypeptide.

56. The method of claim 55, wherein said therapeutic polypeptide is selected from the group consisting of PEDF, CCL4, CXCL13, IL-12, IL-15, and BDNF.

57-82. (canceled)
